(19) United States
(12) Patent Application Publication
(10) Pub. No.: US 2010/0173936 A1
(43) Pub. Date: Jul. 8, 2010

(54) Compositions comprising renin-angiotensin aldosterone system inhibitors and lipoic acid compounds, and the use thereof for the treatment of renin-angiotensin aldosterone system-related disorders

(76) Inventors: Bobby V. Khan, Alpharetta, GA (US); Sampath Parthasarathy, Columbus, OH (US)

Correspondence Address: STITES & HARBISON PLLC, 1199 NORTH FAIRFAX STREET, SUITE 900, ALEXANDRIA, VA, 22314, US

(21) Appl. No.: 12/591,560

(22) Filed: Nov. 23, 2009

Related U.S. Application Data
(60) Provisional application No. 61/118,724, filed on Dec. 1, 2008.

(51) Int. Cl. A61K 31/47; A61K 31/385; A61K 31/20; A61P 3/00; A61K 31/40

(52) U.S. Cl. 514/307; 514/440; 514/433; 514/558; 514/423

(57) ABSTRACT

Compositions are provided which can be useful in treating a renin-angiotensin aldosterone system-related disorder. These compositions include renin-angiotensin aldosterone system inhibitors and lipoic acid compounds, as well as other therapeutic agents, and are useful in treating hypertension, stroke, metabolic syndrome, or other renin-angiotensin aldosterone system-related disorders in a subject. The compositions are also useful in improving vasodilation, reducing proteinuria, and reducing insulin resistance in a subject. Pharmaceutical compositions and methods of treatment using the compositions are further provided.

### CROSS-REFERENCE TO RELATED APPLICATIONS

The present application claims the benefit of U.S. Provisional Application Ser. No. 61/118,724, filed Dec. 1, 2008, the entire disclosure of which is incorporated herein by reference.

### FIELD OF THE INVENTION

The present invention relates to compositions and methods for the treatment of a renin-angiotensin aldosterone system (RAAS)-related disorder. In particular, the present invention relates to compositions including a RAAS inhibitor and a lipoic acid compound that are useful in the treatment of RAAS-related disorders, such as hypertension, diabetes mellitus, target organ damage related to diabetes mellitus, atherosclerosis, coronary heart disease, angina, stroke, renal disorders, Reynaud's disease, metabolic syndrome, obesity, impaired glucose tolerance, and dyslipidemia. In addition, the present invention relates to the use of a composition including a RAAS inhibitor and a lipoic acid compound in improving vasodilation, reducing proteinuria, and reducing insulin resistance in subjects in need of such treatment.

### BACKGROUND OF THE INVENTION

In the United States and other countries, hypertension, stroke, and other disorders related to the renin-angiotensin aldosterone system (RAAS) are a major cause of widespread morbidity and mortality, causing great hardship and economic loss to millions of people throughout the world. It has been estimated that nearly 600 million people worldwide are affected with hypertension, with nearly 50 million of those individuals residing in the United States. Furthermore, it has also been estimated that hypertension alone resulted in an annual expenditure of $66.4 billion in the United States alone in 2007.

Despite the widespread hardship and economic consequences associated with hypertension and other RAAS-related disorders, adequate and appropriate treatment of these disorders has still remained elusive for many individuals as the etiology of these disorders is often multi-factorial. For example, pro-inflammatory mechanisms are thought to be a hallmark of many RAAS-related disorders, such as hypertension and diabetes; however, those findings of inflammation are often exacerbated by the increasing prevalence of obesity worldwide. As another example, metabolic syndrome, a RAAS-related disorder that has reached epidemic proportions over the last decade, often includes multiple components such as abnormal glucose levels, blood pressure, and lipid metabolism (12,46). Further, it has also been observed that individuals displaying multiple components of the metabolic syndrome are at a considerable risk for developing other RAAS-related disorders, including a 2 to 4 fold increased risk of stroke, a 2 to 3 fold increased risk of end-stage renal disease, and a 3 to 4 fold increased risk of myocardial infarction (12). Additionally, recent evidence has indicated that there is a relationship between the etiology of many RAAS-related disorders and oxidative stress and inflammation.

To date, however, and regardless of the increasing amount of evidence that oxidative stress and inflammation play a significant role in the development and pathology of RAAS-related disorders, angiotensin-converting enzyme (ACE) inhibitors and angiotensin II receptor blockers (ARBs) continue to be regarded as preferred agents for the treatment of RAAS-related disorders. It has been known for a number of years that ACE cleaves a C-terminal histidine-leucine dipeptide from the 10 amino acid angiotensin I to generate angiotensin II, which is then able to mediate a variety of physiological responses by binding to an angiotensin II receptor. For example, in addition to the common vasoconstrictive action of angiotensin II, which can lead to increased blood pressure and hypertension, the physiologic effects of angiotensin II also include: ventricular remodeling of the heart, which may lead to ventricular hypertrophy and congestive heart failure; increased free radical generation in blood vessels; stimulation of the adrenal cortex to release aldosterone, which subsequently leads to increases in blood volume and increases in blood pressure; and, stimulation of the posterior pituitary to release vasopressin (also known as anti-diuretic hormone, ADH) which acts on the kidneys to increase water retention. Further, angiotensin II has also been implicated as having multiple effects on inflammation, as well as atherosclerotic plaque development and progression (33, 35, 36).

In light of these wide-ranging effects, the RAAS has thus been implicated extensively in the pathogenesis of many disorders including hypertension, diabetes mellitus, target organ damage related to diabetes mellitus, atherosclerosis, coronary heart disease, angina, stroke, renal disorders, Reynaud's disease, metabolic syndrome, obesity, impaired glucose tolerance, and dyslipidemia. In this regard, recent evidence also suggests that the activation of the RAAS within adipose tissue may represent a link between glucose tolerance, hypertension, and obesity (13). Accordingly, and because angiotensin II is thought to mediate many of the symptoms observed in these disorders, blocking the ability of angiotensin II to bind to its receptors or inhibiting ACE activity thus has great therapeutic potential for the treatment of these disorders. Indeed, ACE inhibitors are currently approved for the treatment of high blood pressure (hypertension) and are also widely prescribed for the treatment of diabetes with target organ damage, systolic heart failure, acute coronary syndrome, and for treatment following a heart attack. The use of ACE inhibitors in these clinical conditions is considered necessary to meet the standard of care as they have been shown to improve clinical outcomes, independent of their blood pressure-lowering effects. However, prescription of ACE inhibitors, or ARBs, for the treatment of these various disorders still largely ignores the underlying oxidative stress and inflammation that accompanies many, if not all, of these disorders. As such, individuals diagnosed with RAAS-related disorders must rely on additional medications to treat the underlying inflammation and oxidative stress.

Currently, a number of anti-inflammatory agents and antioxidants are available, or are naturally-occurring, and are capable of reducing the amount of oxidative stress or inflammation in patients. In plants and animals, one such agent is alpha lipoic acid. Alpha lipoic acid, also known as thioctic acid, is a naturally-occurring 8-carbon fatty acid that is synthesized by plants and animals, including humans, and serves several important functions in the body. Alpha lipoic acid contains two sulfur atoms that are normally found in an oxidized, disulphide form, but which can be reduced to form thiols. This feature allows forms of alpha lipoic acid, such as the lipomide form of alpha lipoic acid, to function as a cofactor for several important enzymes as well as a potent antioxidant. As a potent antioxidant, alpha lipoic acid can scavenge various free radicals and oxidants including hydroxyl radicals, singlet oxygens, peroxynitrite, and hypochlorous acid. Because these free radicals have been implicated in the pathophysiology of many chronic diseases, it is believed that the pharmacotherapeutic effects of alpha lipoic acid are largely due to its antioxidant properties. In addition to its antioxidant properties, however, alpha lipoic acid is also a potent anti-inflammatory reagent. Alpha lipoic acid inhibits the activation of IKK/NF-κB signaling which plays a central role in inflammatory response. Furthermore, a recent report has demonstrated that alpha lipoic acid inhibited atherosclerotic lesion development, due at least in part to its anti-inflammatory effect (51).

Although certain health benefits have been attributed to the administration of exogenous alpha lipoic acid, alpha lipoic acid still continues to be largely viewed as only a nutraceutical supplement with the remainder of its underlying health benefits yet to be fully realized. Furthermore, it remains unknown as to how the structure of alpha lipoic acid can be varied such that a composition could be formulated to obtain the maximum benefits associated with a lipoic acid compound and also be useful in treating an RAAS-related disorder. Indeed, to date, a sufficient lipoic acid compound has failed to be combined with an ACE inhibitor or an ARB, such that the beneficial properties of lipoic acid and those of ACE inhibitors or ARBs could be combined into one composition that is capable of exhibiting a variety of multi-functional therapeutic effects by targeting multiple RAAS-related disorders, and their underlying causes, with minimal toxicity.

Accordingly, a composition that combined a lipoic acid compound with an inhibitor of the RAAS such as ACE inhibitor or an ARB would be highly desirable and potentially very beneficial in treating a variety of disorders related to the action of the RAAS, especially those where the underlying causes are often multi-factorial.

### SUMMARY OF THE INVENTION

It is thus an object of the present invention to provide compositions including an inhibitor of the renin-angiotensin aldosterone system (RAAS), such as an angiotensin-converting enzyme (ACE) inhibitor or an angiotensin II receptor blocker (ARB), and a lipoic acid compound which can be utilized in methods of treating RAAS-related disorders.

It is also an object of the present invention to provide methods for treating a RAAS-related disorder, such as hypertension, diabetes mellitus, target organ damage related to diabetes mellitus, atherosclerosis, coronary heart disease, angina, stroke, renal disorders, Reynaud's disease, metabolic syndrome, obesity, impaired glucose tolerance, and dyslipidemia, wherein a subject in need of treatment is administered an effective amount of a composition of the present invention to thereby treat the RAAS-related disorder.

It is another object of the present invention to provide a method improving vasodilation, such as flow-mediated vasodilation, wherein a subject in need of treatment is administered an effective amount of a composition of the present invention to thereby improve vasodilation in the subject.

It is a further object of the present invention to provide a method of reducing proteinuria in a subject by administering an effective amount of a composition of the present invention, which can reduce an amount of urinary albumin or a ratio of urinary albumin to serum creatinine, to thereby reduce proteinuria in the subject.

It is still further an object of the present invention to provide a method of reducing insulin resistance, such as by increasing insulin receptor sensitivity, wherein a subject in need of treatment is administered an effective amount of a composition of the present invention to thereby reduce insulin resistance in the subject.

These and other objects are provided by virtue of the present invention which comprises compositions that include a RAAS inhibitor and a lipoic acid compound. In a preferred embodiment of the present invention, compositions are provided that include a RAAS inhibitor and a lipoic acid compound selected from the group consisting of the following Formulas (I) and (II), or pharmaceutically-acceptable salts or solvates thereof:

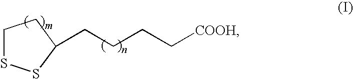

wherein: m is an integer from 1 to 2; and n is an integer from 1 to 5; and

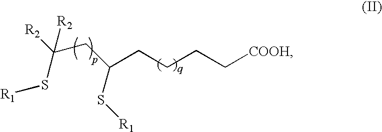

wherein: p is an integer from 1 to 2; q is an integer from 1 to 5; R₁ is selected from the group consisting of H, methyl, NO, and acetyl; and R₂ is selected from the group consisting of H, methyl, and tert-butyl.

In another preferred embodiment of the invention, a composition is provided wherein m is 2 in a lipoic acid compound of Formula (I). In another embodiment, a composition of the present invention is provided where n is an integer from 2 to 5 in a lipoic acid compound of Formula (I).

In yet another preferred embodiment of the present invention, compositions including a RAAS inhibitor and a lipoic acid compound of the foregoing Formulas (I) and (II) are provided, where the RAAS inhibitor is either an angiotensin-converting enzyme (ACE) or an angiotensin II receptor blocker (ARB). Numerous ACE inhibitors can be used in accordance with compositions of the present invention including, but not limited to, benazepril, captopril, cilazapril, enalapril, enalaprilat, fosinopril, lisinopril, moexipril, perindopril, quinapril, ramipril, trandolapril, and zofenopril. Similarly, numerous ARBs can also be used in accordance with the compositions of the present invention including, but not limited to, candesartan, eprosartan, irbesartan, telmisartan, valsartan, losartan, and olmesartan. Each of these ACE inhibitors and ARBs can effectively be combined with a lipoic acid compound of the present invention to produce a composition that is useful in treating a RAAS-related disorder.

In still another preferred embodiment of the present invention, compositions that include a RAAS inhibitor and a lipoic acid compound of the foregoing Formulas (I) and (II) are provided that further comprise one or more additional agents that are useful in treating a RAAS-related disorder. In one further preferred embodiment, a composition of the present invention is provided that further includes a statin, such as atorvastatin, fluvastatin, lovastatin, mevastatin, pitavastatin, pravastatin, rosuvastatin, and simvastatin. In other embodiments, a composition of the present invention is provided that further includes an anti-inflammatory agent, an agent that inhibits the absorption of fatty acids, or combinations thereof.

In addition, the present invention provides pharmaceutical compositions wherein the compositions of the present invention further comprise a pharmaceutically-acceptable vehicle, carrier, or excipient, or are in a sustained-release formulation.

These embodiments and other alternatives and modifications within the spirit and scope of the presently-disclosed invention will become readily apparent to those of ordinary skill in the art after a study of the description, Figures, and non-limiting Examples in this document.

### BRIEF DESCRIPTION OF THE DRAWING FIGURES

FIG. 1 is a graph showing the amount of urinary albumin and the ratio of urinary albumin to creatinine in urine samples obtained from diabetic hypertensive subjects prior to treatment (Pretreatment) and subsequent to treatment with either 40 mg/day of quinapril (Qui), or treatment with a combination of 40 mg/day of quinapril and 600 mg/day of alpha lipoic acid (Qui/ALA).

FIG. 2 is a graph showing the amount of flow-mediated dilation observed in diabetic hypertensive subjects prior to treatment (Pretreatment) and subsequent to treatment with either 40 mg/day of quinapril (Qui), or treatment with a combination of 40 mg/day of quinapril and 600 mg/day of alpha lipoic acid (Qui/ALA).

FIG. 3 is a graph showing indices of insulin resistance, obtained from a homeostasis model of assessment of insulin resistance (HOMA-IR), that are observed in diabetic hypertensive subjects prior to treatment (Pretreatment) and subsequent to treatment with either 40 mg/day of quinapril (Qui), or treatment with a combination of 40 mg/day of quinapril and 600 mg/day of alpha lipoic acid (Qui/ALA).

FIG. 4 is a graph showing serum levels of the inflammatory molecule PAI-1 in subjects diagnosed with metabolic syndrome and treated with either 20 mg/day of quinapril, 300 mg/day of alpha lipoic acid, or with a combination of 20 mg/day of quinapril and 300 mg/day of alpha lipoic acid.

FIG. 5 is a graph showing serum levels of the inflammatory molecule VCAM-1 in subjects diagnosed with metabolic syndrome and treated with either a placebo, 20 mg/day of quinapril, 300 mg/day of alpha lipoic acid, or with a combination of 20 mg/day of quinapril and 300 mg/day of alpha lipoic acid.

FIG. 6 is a graph showing the amount of endothelial dilation that is observed in subjects diagnosed with metabolic syndrome and treated with either a placebo or with a combination of 20 mg/day of quinapril and 300 mg/day of alpha lipoic acid.

### DETAILED DESCRIPTION OF THE PREFERRED EMBODIMENTS

In accordance with the present invention, compositions and methods for treating a renin-angiotensin aldosterone system (RAAS)-related disorder are provided. In particular, the present invention provides compositions that include a RAAS inhibitor and a lipoic acid compound and are useful in treating RAAS-related disorders, such as metabolic syndrome. Further these compositions are also useful in improving vasodilation, reducing proteinuria, and reducing insulin resistance in subjects in need of such treatment. In some embodiments, the compounds can be administered as part of a pharmaceutical composition, such as in a sustained-release formulation, to thereby treat a RAAS-related disorder in a subject.

In one of the preferred embodiments of the present invention, a composition useful in the invention comprises a RAAS inhibitor and a lipoic acid compound selected from the groups consisting of the following Formulas (I) and (II):

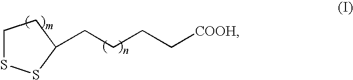

wherein: m is an integer from 1 to 2; and n is an integer from 1 to 5; and

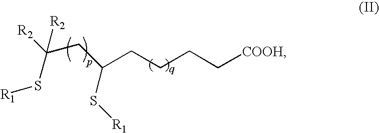

wherein: p is an integer from 1 to 2; q is an integer from 1 to 5; R₁ is selected from the group consisting of H, methyl, NO, and acetyl; and R₂ is selected from the group consisting of H, methyl, and tert-butyl.

As used herein, the term “lipoic acid compound” refers to compounds having a general formula of Formula (I) or Formula (II) above. These compounds will include both alpha lipoic acid (i.e., when m is 1 and n is 1 in Formula (I) above) and dihydrolipoic acid (i.e., when R₁ and R₂ are both hydrogen atoms (H), p is 1, and q is 1 in Formula (II) above), as well as other oxidized and reduced forms of lipoic acid as indicated by Formulas (I) and (II), respectively. For example, in certain embodiments of the lipoic acid compound of Formula (I), m can be 1 or 2 such that a five-membered ring structure can be provided and/or n can an integer from 1 to 5 such that the length of the alkyl chain in a compound of Formula (I) can be increased by 1, 2, 3, or 4 additional carbon atoms. As another example, in certain embodiments of the lipoic acid compound of Formula (II), p can be 1 or 2, or n can an integer from 1 to five such that the length of the alkyl chain in a compound of Formula (II) can be increased by 1, 2, 3, or 4 additional carbon atoms. Additionally, in certain embodiments of the compound of Formula (II), R₁ can be varied such that the resulting lipoic acid compound of Formula (II) includes one or two hydrogen atoms (H), methyl groups (—CH₃), —NO groups, or acetyl groups (—COCH₃), or R₂ can be varied such that the resulting lipoic acid compound of Formula (II) includes one or two hydrogen atoms, methyl groups, or tert-butyl groups.

With further regard to the lipoic acid compounds of Formulas (I) and (II), it is also noted that m, n, p, q, R₁, and R₂ are independent from one another. For example, in certain embodiments of the lipoic acid compound of Formula (I), a lipoic acid compound can be provided where m is 1 and n is 2. As another example, in certain embodiments of the lipoic acid compound of Formula (II), a lipoic acid compound can be provided where p is 1, q is 2, each R₁ is H, and each R₂ is CH₃.

In one preferred embodiment of the invention, a lipoic acid compound of Formula (I) is provided where m equals 1 and n equals 3, as shown by the following Formula (III):

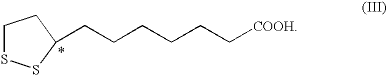

In another preferred embodiment of the invention, a lipoic acid compound of Formula (I) is provided where m equals 2 and n equals 1, as shown be the following Formula (IV):

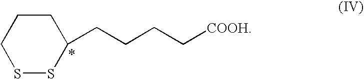

In yet another preferred embodiment of the invention, a lipoic acid compound of Formula (I) is provided where m equals 2 and n equals 4, as shown by the following Formula (V):

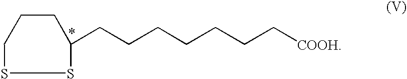

In still another preferred embodiment of the invention, a lipoic acid compound of Formula (II) is provided where p equals 2, q equals 1, both R₁ groups are H, and both R₂ groups are H, as shown by the following Formula (VI):

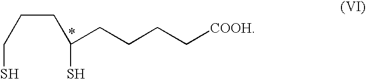

In other preferred embodiments of the invention, a lipoic acid compound of Formula (II) is provided where p equals 1, q equals 1, both R₁ groups are acetyl groups, and both R₂ groups are H, as shown by the following Formula (VII):

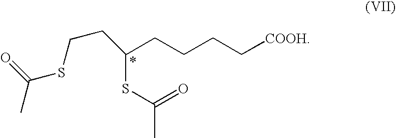

In another embodiment of the invention, a lipoic compound of Formula (II) is provided where p equals 2, q equals 1, both R₁ groups are NO groups, and both R₂ groups are H, as shown by the following Formula (VIII):

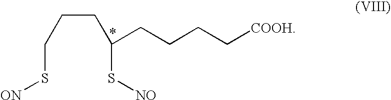

In other embodiments of the invention, a lipoic acid compound of Formula (II) is provided where p equals 2, q equals 1, both R₁ groups are NO groups, one R₂ group is H, and another R₂ group is a tert-butyl group, as shown by the following Formula (IX):

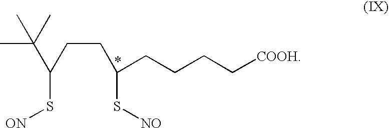

In further embodiments of the invention, a lipoic acid compound of Formula (II) is provided where p equals 1, q equals 1, both R₁ groups are methyl groups, one R₂ group is H, and another R₂ group is a methyl group, as shown by the following Formula (X):

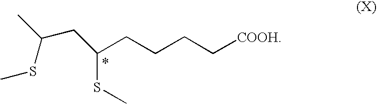

In yet another embodiment of the invention, a lipoic acid compound of Formula (II) is provided where p equals 1, q equals 1, both R₁ groups are NO groups, and both R₂ groups are methyl groups, as shown by the following Formula (XI):

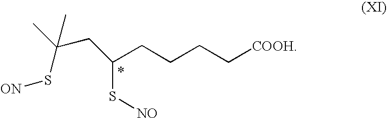

In some embodiments of the lipoic acid compounds of Formulas (I) and (II), the lipoic acid compounds can include a stereo-isomeric carbon atom as shown by (*) in Formulas (III)-(IV) above and the chemical structures provided below. As such, in some embodiments of the presently-disclosed lipoic acid compounds, the compounds are inclusive of L-, D-, and D,L-isomers.

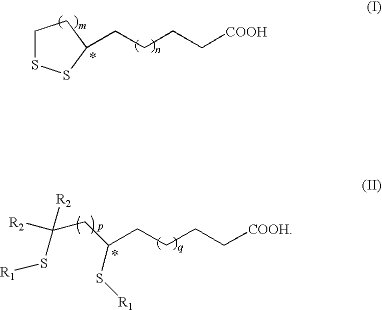

In addition, and as indicated above, the lipoic acid compounds included herein are described with reference to formulas where one or more additional moieties can be incorporated into the core structure. In these embodiments, reference to the lipoic acid compounds of the present invention can include stereoisomers of the one or more moieties of the compounds. Such stereoisomers are representative of some embodiments of the lipoic acid compounds; however, the formulas and reference to the formulas disclosed herein are intended to encompass all active stereoisomers of the depicted lipoic acid compounds. Furthermore, the lipoic acid compounds of the presently-disclosed subject matter can, in some embodiments, contain one or more additional asymmetric carbon atoms, other than those indicated above, and can exist in raecemic and optically active forms. All of these other forms are contemplated to be within the scope of the present invention. As such, the lipoic acid compounds of the present invention can exist in stereoisomeric forms and the products obtained can thus be mixtures of the isomers.

In accordance with the present invention, all of the lipoic acid compounds described herein can be provided in the form of a pharmaceutically-acceptable salt or solvate, as would be recognized by one skilled in the art. A salt can be formed using a suitable acid and/or a suitable base. Suitable acids that are capable of forming salts with the lipoic acid compounds of the present invention include inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid, or the like. Suitable bases capable of forming salts with the lipoic acid compounds of the present invention include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine, and the like), and optionally substituted ethanolamines (e.g., ethanolamine, diethanolamine, and the like).

As used herein, the term “solvate” refers to a complex or aggregate formed by one or more molecules of a solute, e.g., a lipoic acid compound of the present invention or a pharmaceutically-acceptable salt thereof, and one or more molecules of a solvent. Such solvates are typically crystalline solids having a substantially fixed molar ratio of solute and solvent. Representative solvents include, but are not limited to, water, methanol, ethanol, isopropanol, acetic acid, and the like. When the solvent is water, the solvate formed is a hydrate. As such, the term “pharmaceutically-acceptable salt or solvate thereof” is intended to include all permutations of salts and solvates, such as a solvate of a pharmaceutically-acceptable salt of the present lipoic acid compounds.

In yet a further embodiment of the compositions of the present invention, and as described further below, pharmaceutical compositions are provided which comprise the compositions described herein and a pharmaceutically acceptable vehicle, carrier or excipient. For example, solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, but are not limited to, microcrystalline cellulose, corn starch, sodium starch glycolate, and alginic acid. Tablet binders that can be used include acacia, methylcellulose, sodium carboxymethylcellulose, polyvinylpyrrolidone (POVIDONE™), hydroxypropyl methylcellulose, sucrose, starch, and ethylcellulose. Lubricants that can be used include magnesium stearates, stearic acid, silicone fluid, talc, waxes, oils, and colloidal silica. Further, the solid formulations can be uncoated or they can be coated by known techniques to delay disintegration and absorption in the gastrointestinal tract and thereby provide a sustained/extended action over a longer period of time. For example, glyceryl monostearate or glyceryl distearate can be employed to provide a sustained-/extended-release formulation. Numerous techniques for formulating sustained release preparations are known to those of ordinary skill in the art and can be used in accordance with the present invention, including the techniques described in the following references: U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; and WO 98/47491, each of which is incorporated herein by this reference.

In one preferred embodiment, a sustained-release formulation of a composition of the present invention is provided that utilizes a polyanhydride-based technology. As will be recognized by those skilled in the art, polyanhydrides are a distinctive class of polymers for drug delivery because of their biodegradability and biocompatibility properties. In some embodiments, the release rate of polyanhydride-based formulations can be tuned over several folds by incorporating changes in the polymer structure. As such, in some embodiments of the sustained-release formulations of the presently-described compositions, the polymers employed to provide a sustained-release formulation are selected from poly[1,3-bis(p-carboxyphenoxy)propane, poly[1,3-bis(p-carboxyphenoxy)hexane-co-sebacic anhydride], poly[1,3-bis(p-carboxyphenoxy)methan-co-sebacic anhydride], and poly(fumaric anhydride). Apart from polyanhydride based formulations, in some embodiments, chitosan-based control release technology can be employed to provide a sustained-release formulation, as described further below.

Furthermore, liquid formulations of the compounds for oral administration can be prepared in water or other aqueous vehicles, and can contain various suspending agents such as methylcellulose, alginates, tragacanth, pectin, kelgin, carrageenan, acacia, polyvinylpyrrolidone, and include solutions, emulsions, syrups, and elixirs containing, together with the active components of the composition, wetting agents, sweeteners, and coloring and flavoring agents.

Various liquid and powder formulations can also be prepared by conventional methods for inhalation into the lungs of the subject to be treated. For example, the compositions can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. Capsules and cartridges of, for example, gelatin for use in an inhaler or insufflator may be formulated containing a powder mix of the composition and a suitable powder base such as lactose or starch.

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol), and the like. For intravenous injections, water soluble versions of the compounds can be administered by the drip method, whereby a formulation including a pharmaceutical composition of the present invention and a physiologically-acceptable excipient is infused. Physiologically-acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the compound can be prepared and administered as a suspension in an aqueous base or a pharmaceutically-acceptable oil base, such as an ester of a long chain fatty acid, (e.g., ethyl oleate).

In addition to the formulations described above, the compositions of the present invention can also be formulated as rectal compositions, such as suppositories or retention enemas, e.g., containing conventional suppository bases such as cocoa butter or other glycerides. Further, the compositions can also be formulated as a depot preparation by combining the compositions with suitable polymeric or hydrophobic materials (for example as an emulsion in an acceptable oil) or ion exchange resins, or as sparingly soluble derivatives, for example, as a sparingly soluble salt.

In some embodiments of the present invention, the compounds of the present invention may be incorporated into a nanoparticle. A nanoparticle within the scope of the invention is meant to include particles at the single molecule level as well as those aggregates of particles that exhibit microscopic properties. Methods of using and making a nanoparticle that incorporates a composition of interest are known to those of ordinary skill in the art and can be found following references: U.S. Pat. Nos. 6,395,253, 6,387,329, 6,383,500, 6,361,944, 6,350,515, 6,333,051, 6,323,989, 6,316,029, 6,312,731, 6,306,610, 6,288,040, 6,272,262, 6,268,222, 6,265,546, 6,262,129, 6,262,032, 6,248,724, 6,217,912, 6,217,901, 6,217,864, 6,214,560, 6,187,559, 6,180,415, 6,159,445, 6,149,868, 6,121,005, 6,086,881, 6,007,845, 6,002,817, 5,985,353, 5,981,467, 5,962,566, 5,925,564, 5,904,936, 5,856,435, 5,792,751, 5,789,375, 5,770,580, 5,756,264, 5,705,585, 5,702,727, and 5,686,113, each of which is incorporated herein by this reference.

Nanoparticles are frequently regarded as solid colloidal particles ranging in size from 10 nm to 1 μm, and can be built from macromolecular assemblies, in which an active compound or agent (e.g., a lipoic acid compound or a RAAS inhibitor) is dissolved, entrapped, encapsulated, or adsorbed or attached to the external interface to provide kinetic stability and rigid morphology. In some embodiments of the present invention, a bio-polymer-based nanoparticle formulation is utilized for efficient delivery of a composition of the presently-disclosed subject matter. In some embodiments, a formulation can be provided that utilizes chitosan/polyguluronate nanoparticles, poly(D,L-lactic acid)/ethyl acetate-based nanoparticles, PLGA-, PLGA:poloxamer-, or PLGA:poloxamine/dichloromethane-mediated nanoparticles, PEGylated polymeric micelles, or nanoparticles of albumin. As will be recognized by those of skill in the art, the preparation of nanoparticles as a composition vehicle will depend on the types of biopolymers employed in the process.

In one preferred embodiment of the present invention, a nanoparticle formulation can be provided that is derived from a chitosan/polyguluronate combination. Chitosan is a naturally existing polysaccharide composed of glucosamine and N-acetylglucosamine residues and can be derived by partial deacetylation of chitin, which is generally obtained from crustacean shells. Chitosan is known to be a biocompatible, low toxic, low immunogenic, and degradable by enzymes. In this regard, a nanoparticle formulation of the present invention can be prepared by first dissolving chitosan glutamate in a suitable buffer, and, similarly, dissolving polyguluronate in a sodium sulfate buffer. The solutions can then be filtered through a micro-filter, and the nanoparticle formulations can then be prepared by adding the chitosan solution to an equal volume of the polyguluronate solution and then incubating the particles room temperature. In this regard, to incorporate a composition of the present invention into the nanoparticles, a desired amount of the composition, in a polar solvent, can be first added to the polyguluronate solution, and then the mixture can be combined with the chitosan solution. The resulting nanoparticles can then be incubated at room temperature before use or further analysis (see, e.g., Hoffman A S, The origins and evolution of “controlled” drug delivery systems, Journal of Controlled Release, 132 (2008), 153-163).

In some embodiments of the compositions of the present invention, the RAAS inhibitor that is included in the composition is selected from the group consisting of angiotensin-converting enzyme inhibitors and angiotensin II receptor blockers. Angiotensin-converting enzyme (ACE) is a peptidylcarboxypeptidase, which catalyzes the cleavage of the histidine-leucine dipeptide at the carboxy-terminus of the inactive decapeptide angiotensin I to form angiotensin II, and is also responsible for the deactivation of bradykinase. Once the dipeptide has been cleaved from the carboxy-terminus of angiotensin I and angiotensin II has been formed, angiotensin II is then able to mediate a variety of responses, as described further below, by binding to and activating the angiotensin receptors AT₁ and AT₂, which subsequently mediate a variety of physiological responses with the RAAS. As such, the term “RAAS inhibitors,” as used herein, refers to agents that are capable of reducing the activity of angiotensin II within the RAAS. The term “RAAS inhibitor” is thus inclusive of agents that are capable of inhibiting the conversion of angiotensin Ito angiotensin II, e.g., ACE inhibitors, as well as agents that are capable of blocking the binding of angiotensin II to its receptors and thus reducing the activation of the receptors, e.g., angiotensin II receptor blockers or “ARBs”, which may also be referred to as angiotensin II receptor antagonists, AT₁-receptor antagonists, or sartans. Numerous ACE inhibitors and ARBs are known to those of ordinary skill in the art and can be used in accordance with the compositions of the present invention. In some embodiments, the RAAS inhibitor is an ACE inhibitor that is selected from the group consisting of benazepril, captopril, cilazapril, enalapril, enalaprilat, fosinopril, lisinopril, moexipril, perindopril, quinapril, ramipril, trandolapril, and zofenopril. In other embodiments, the RAAS inhibitor is an ARB that is selected from the group consisting of candesartan, eprosartan, irbesartan, telmisartan, valsartan, losartan, and olmesartan.

In some embodiments of the presently-disclosed compositions, which include a RAAS inhibitor and a lipoic acid compound of Formula (I) or (II), the compositions can further include one or more additional agents that are useful in treating a RAAS-related disorder. For example, in certain embodiments of the present invention, a statin is further combined with a RAAS inhibitor and a lipoic acid compound of Formula (I) or (II) to produce a composition of the present invention. Various statins (i.e., HMG-CoA reductase inhibitors) are known to those of ordinary skill in the art as agents that are capable inhibiting the HMG-CoA reductase enzyme and thus decreasing cholesterol synthesis and increasing synthesis of low-density lipoprotein (LDL) receptors, which then results in an increased clearance of LDLs from the blood stream of a subject. In certain embodiments of the compositions described herein, the statin that is combined with a RAAS inhibitor and a lipoic acid compound of Formula (I) or (II) can be selected from atorvastatin, fluvastatin, lovastatin, mevastatin, pitavastatin, pravastatin, rosuvastatin, and simvastatin. Each of these statins can be combined with a composition of the present invention and be useful in treating a RAAS-related disorder.

In certain embodiments of the present compositions, a composition that includes a RAAS inhibitor and a lipoic acid compound of the present invention is provided that further includes an anti-inflammatory agent. Examples of anti-inflammatory agents which may be used in accordance with the compositions of the present invention include, but are not limited to, classic non-steroidal anti-inflammatory agents (NSAIDS), such as aspirin, diclofenac, indomethacin, sulindac, ketoprofen, flurbiprofen, ibuprofen, naproxen, piroxicam, tenoxicam, tolmetin, ketorolac, oxaprosin, mefenamic acid, fenoprofen, nambumetone (relafen), acetaminophen, and combinations thereof; COX-2 inhibitors, such as nimesulide, flosulid, celecoxib, rofecoxib, parecoxib sodium, valdecoxib, etoricoxib, etodolac, meloxicam, and combinations thereof; glucocorticoids, such as hydrocortisone, cortisone, prednisone, prednisolone, methylprednisolone, meprednisone, triamcinolone, paramethasone, fluprednisolone, betamethasone, dexamethasone, fludrocortisone, desoxycorticosterone, rapamycin; or others or analogues of these agents or combinations thereof.

In other embodiments of the present compositions, an agent that inhibits the absorption of fatty acids, such as ezetimibe, sulfated polysaccharides, oleayl alcohols, or lecithin, can be further combined with a composition of the present invention. Agents that inhibit the absorption of fatty acids can also be combined with one or more additional agents, such as an anti-inflammatory agent or a statin, to produce a composition of the present invention that includes a RAAS inhibitor, a lipoic acid compound, and one or more of the additional agents, such that a further composition can be provided that is useful in treating a RAAS-related disorder.

With further regard to the compositions of the present invention, it is contemplated that each of the lipoic acid compounds or agents included in a composition of the present invention are further inclusive of derivatives of those compounds or agents. Exemplary derivatives of an alpha lipoic acid compound in accordance with the present invention are included in Formulas (I) and (II) above; however, it is noted that the present compositions can also include further derivatives of the agents and lipoic acid compounds of the present invention, including derivatives of RAAS inhibitors, derivatives of statins, derivatives of anti-inflammatory agents, derivatives of agents that inhibit the absorption of fatty acids, and combinations thereof. As used herein, the term “derivative” refers to a chemically or biologically modified version of a chemical compound that is structurally similar to the parent compound and derivable from that parent compound. A “derivative” differs from an “analogue” in that a parent compound can be the starting material to generate a “derivative,” whereas the parent compound may not necessarily be used as the starting material to generate an “analogue.” Additionally, a derivative may or may not have different chemical or physical properties of the parent compound. For example, the derivative may be more hydrophilic or it may have altered reactivity as compared to the parent compound. In this regard, derivatization (i.e., modification) may involve substitution of one or more moieties within the molecule (e.g., a change in functional group). For example, a hydrogen may be substituted with a halogen, such as fluorine or chlorine, or, as another example, a hydroxyl group (—OH) may be replaced with a carboxylic acid moiety (—COOH).

As used herein, the term “derivative” also includes conjugates and prodrugs (i.e., chemically modified derivatives which can be converted into the original compound under physiological conditions) of a parent compound. For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). Further information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443, each of which is incorporated herein by this reference.

In accordance with the present invention, methods for treating a RAAS-related disorder using the presently-disclosed compositions are also provided. In one preferred embodiment, a method for treating a RAAS-related disorder is provided that comprises administering to a subject an effective amount of a composition of the present invention, which includes a RAAS inhibitor and a lipoic acid compound of Formula (I) or (II), or pharmaceutically-acceptable salts or solvates thereof, to thereby treat the RAAS-disorder in the subject.

As used herein, the terms “treatment” or “treating” relate to any treatment of a RAAS-related disorder, including but not limited to prophylactic treatment and therapeutic treatment. As such, the terms “treatment” or “treating” include, but are not limited to: preventing a RAAS-related disorder or the development of a RAAS-related disorder; inhibiting the progression of a RAAS-related disorder; arresting or preventing the further development of a RAAS-related disorder; reducing the severity of a RAAS-related disorder; ameliorating or relieving symptoms associated with a RAAS-related disorder; and causing a regression of a RAAS-related disorder or one or more of the symptoms associated with a RAAS-related disorder.

The term “renin-angiotensin aldosterone system-related disorder” or “RAAS-related disorder” is used herein to refer to disorders that are caused by, at least in part, or exacerbated by the actions of the renin-angiotensin aldosterone system. As noted herein, angiotensin II is a central mediator of the action of the RAAS and mediates a variety of effects in subjects including: vasoconstriction, which can lead to increased blood pressure and hypertension; ventricular remodeling of the heart, which may lead to ventricular hypertrophy and congestive heart failure; increased free radical generation in blood vessels; stimulation of the adrenal cortex to release aldosterone, which subsequently leads to increased blood volume and hence an increase in blood pressure; stimulation of the posterior pituitary to release vasopressin (also known as anti-diuretic hormone, ADH) which also acts on the kidneys to increase water retention; increased inflammation and expression of various inflammatory genes, which can lead to inflammation in an affected subject; endothelial dysfunction; and vascular plaque development. In addition to the actions of angiotensin II, the activation of the RAAS has also been implicated in, for example: reactive oxygen species development; activation and adhesion of monocytes to vascular walls; increased uptake of modified low density lipoprotein into monocytes, which creates atherogenic “foam cells;” and reduced endothelial synthesis of nitric oxide. Given these wide-ranging effects of the RAAS, and in particular Angiotensin II, the RAAS has thus been implicated in a variety of disorders including, but not limited to, hypertension, diabetes mellitus, target organ damage related to diabetes mellitus, atherosclerosis, coronary heart disease, angina, stroke, renal disorders, Reynaud's disease, metabolic syndrome, obesity, impaired glucose tolerance, and dyslipidemia. See, e.g., Ferrario C M, Role of Angiotensin II in Cardiovascular Disease: Therapeutic Implications of More Than a Century of Research, J Renin Angiotensin Aldosterone Syst, 2006; 7: 3-14, which is incorporated herein by reference. As such, in certain embodiments, the RAAS-related disorder is selected from hypertension, diabetes mellitus, target organ damage related to diabetes mellitus, atherosclerosis, coronary heart disease, angina, stroke, renal disorders, Reynaud's disease, metabolic syndrome, obesity, impaired glucose tolerance, and dyslipidemia.

For administration of a therapeutic composition as disclosed herein, conventional methods of extrapolating human dosage based on doses administered to a murine animal model can be carried out using the conversion factor for converting the mouse dosage to human dosage: Dose Human per kg=Dose Mouse per kg×12 (Freireich, et al., (1966) Cancer Chemother Rep. 50:219-244). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

Suitable methods for administering a therapeutic composition in accordance with the methods of the present invention include, but are not limited to, systemic administration, parenteral administration (including intravascular, intramuscular, intraarterial administration), oral delivery, buccal delivery, rectal delivery, subcutaneous administration, intraperitoneal administration, inhalation, intratracheal installation, surgical implantation, transdermal delivery, local injection, and hyper-velocity injection/bombardment. Where applicable, continuous infusion can enhance drug accumulation at a target site (see, e.g., U.S. Pat. No. 6,180,082).

Regardless of the route of administration, the compounds of the present invention are typically administered in amount effective to achieve the desired response. As such, the term “effective amount” is used herein to refer to an amount of the therapeutic composition (e.g., a compound comprising a RAAS inhibitor, a lipoic acid compound of Formula (I) or (II), and a pharmaceutically vehicle, carrier, or excipient) sufficient to produce a measurable biological response (e.g., a reduction in a blood pressure). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

In certain embodiments of the methods of treating a RAAS-related disorder disclosed herein, the lipoic acid compound and RAAS inhibitor can be combined in a composition at dosage ranges such as those provided in Table 1 below. For example, in some embodiments, a composition of the present invention can be administered once daily to a subject, where the composition includes: 300 mg of a lipoic acid compound and 20 mg of quinapril; 300 mg of a lipoic acid compound and 20 mg of lisinopril; 300 mg of a lipoic acid compound and 20 mg of fosinopril; 600 mg of a lipoic acid compound and 5 mg of ramipril; or, 600 mg of a lipoic acid compound and 10 mg of lisinopril. When a statin is included in a composition of the present invention, the dosage range of the statin can be, for example, about 1 mg to about 100 mg per day. When an anti-inflammatory agent or agent that inhibits absorption of fatty acids is included in a composition of the present invention, the dosage ranges of those agents can include the dosage ranges that would typically be employed for those specific agents. Of course, additional variations of the above-described doses can be utilized in a composition of the present invention to achieve the desired biological response, and can be ascertained by those of ordinary skill in the art of medicine using routine experimentation.

TABLE 1
Exemplary Dosage Ranges.

| Active Ingredient | Dosage Range |
|---|---|
| RAAS Inhibitor | 0.1 mg/day to 100 mg/day |
| | 1 mg/day to 80 mg/day |
| | 5 mg/day to 50 mg/day |
| | 5, 10, or 20 mg/day |
| Lipoic Acid Compound | 1 mg/day to 1000 mg/day |
| | 10 mg/day to 600 mg/day |
| | 100 mg/day to 400 mg/day |
| | 300, 400, 500 or 600 mg/ |
| | day |
| Statin | 1 mg/day to 100 mg/day |
| | 10 mg/day to 80 mg/day |
| | 20 mg/day to 60 mg/day |

For additional guidance regarding formulation and dose, see U.S. Pat. Nos. 5,326,902 and 5,234,933; PCT International Publication No. WO 93/25521; Berkow, et al., (1997) The Merck Manual of Medical Information, Home ed. Merck Research Laboratories, Whitehouse Station, N.J.; Goodman, et al., (2006) Goodman & Gilman's the Pharmacological Basis of Therapeutics, 11th ed. McGraw-Hill Health Professions Division, New York; Ebadi. (1998) CRC Desk Reference of Clinical Pharmacology. CRC Press, Boca Raton, Fla.; Katzung, (2007) Basic & Clinical Pharmacology, 10th ed. Lange Medical Books/McGraw-Hill Medical Pub. Division, New York; Remington, et al., (1990) Remington's Pharmaceutical Sciences, 18th ed. Mack Pub. Co., Easton, Pa.; Speight, et al., (1997) Avery's Drug Treatment: A Guide to the Properties, Choice, Therapeutic Use and Economic Value of Drugs in Disease Management, 4th ed. Adis International, Auckland/Philadelphia; and Duch, et al., (1998) Toxicol. Lett. 100-101:255-263, each of which are incorporated herein by reference.

In certain embodiments of the presently-disclosed methods of treating an RAAS-related disorder, administering a composition of the present invention to the subject increases endothelial function in a blood vessel of the subject. As will be recognized by those skilled in the art, the endothelium is the monolayer of endothelial cells that lines the lumen of all blood vessels. Typically, these cells function as a protective biocompatible barrier between all tissues and the circulating blood, and also function as a selective sieve that facilitates the bidirectional passage of macromolecules and blood gases to and from tissues and blood. Indeed, the strategic location of the endothelium allows it to sense changes in hemodynamic forces and blood-borne signals and respond accordingly by releasing a number of autocrine and paracrine substances. A balanced release of these bioactive factors facilitates vascular homeostasis. Endothelial cell dysfunction, however, such as what occurs in many RAAS-related disorders, disrupts this balance, and thereby predisposes the vessel wall to vasoconstriction, leukocyte adherence, platelet activation, mitogenesis, pro-oxidation, thrombosis, impaired coagulation, vascular inflammation, and plaque development. Disclosed herein, however, are data indicating that an effective amount of a composition of the present invention can be administered to a subject to thereby increase endothelial function in the subject and potentially avoid the adverse events that may otherwise occur with endothelial dysfunction.

Various methods known to those of skilled in the art can be used to assess endothelial function in a blood vessel of a subject. For example, in certain embodiments, vascular endothelial function can be evaluated using a non-invasive, brachial artery reactivity testing (BART) technique, which uses ultrasound to evaluate flow-mediated vasodilatation in the brachial artery. Briefly, that test stimulates the endothelium of the brachial artery in the arm to release nitric oxide, which then causes vasodilatation of the artery. The resulting vasodilatation can then be measured and quantified as a marker of endothelial function.

In other embodiments of the therapeutic methods disclosed herein, administering a composition of the present invention to the subject reduces serum levels of an inflammatory molecule in a subject. As noted herein, recent evidence has indicated that RAAS-related disorders, such as hypertension, are closely related to the amount of oxidative stress and inflammation in a subject. It has been discovered though that by administering a composition of the present invention to a subject affected with an RAAS-related disorder, the serum levels of inflammatory molecules in the subject can be advantageously reduced. In certain embodiments, administering a composition of the present invention reduces levels of the inflammatory molecules plasminogen activator inhibitor-1 (PAI-1), vascular cell adhesion molecule-1 (VCAM-1), leptin, and/or adiponectin in a subject.

Various methods known to those skilled in the art can be used to determine a reduction of serum levels of inflammatory molecules in a subject. For example, in certain embodiments, the amounts of expression of an inflammatory molecule in a subject can be determined by probing for mRNA of the gene encoding the inflammatory molecule (e.g., PAI-1, VCAM-1, leptin, or adiponectin) in a biological sample obtained from the subject (e.g., a tissue sample, a urine sample, a saliva sample, a blood sample, a serum sample, a plasma sample, or sub-fractions thereof) using any RNA identification assay known to those skilled in the art. Briefly, RNA can be extracted from the sample, amplified, converted to cDNA, labeled, and allowed to hybridize with probes of a known sequence, such as known RNA hybridization probes immobilized on a substrate, e.g., array, or microarray, or quantitated by real time PCR (e.g., quantitative real-time PCR, such as available from Bio-Rad Laboratories, Hercules, Calif., U.S.A.). Because the probes to which the nucleic acid molecules of the sample are bound are known, the molecules in the sample can be identified. In this regard, DNA probes for one or more of the mRNAs encoded by the inflammatory genes can be immobilized on a substrate and provided for use in practicing a method in accordance with the present invention.

With further regard to determining levels of inflammatory molecules in samples, mass spectrometry and/or immunoassay devices and methods can be used to measure the inflammatory molecules in samples, although other methods can also be used and are well known to those skilled in the art. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Immunoassay devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377, each of which is hereby incorporated by reference in its entirety.

Any suitable immunoassay can be utilized, for example, enzyme-linked immunoassays (ELISA), radioimmunoassays (RIAs), competitive binding assays, and the like. Specific immunological binding of the antibody to the inflammatory molecule can be detected directly or indirectly. Direct labels include fluorescent or luminescent tags, metals, dyes, radionucleotides, and the like, attached to the antibody. Indirect labels include various enzymes well known in the art, such as alkaline phosphatase, horseradish peroxidase and the like.

The use of immobilized antibodies or fragments thereof specific for the inflammatory molecules is also contemplated by the present invention. The antibodies can be immobilized onto a variety of solid supports, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on a solid support. This strip can then be dipped into the test biological sample and then processed quickly through washes and detection steps to generate a measurable signal, such as for example a colored spot.

Mass spectrometry (MS) analysis can be used, either alone or in combination with other methods (e.g., immunoassays), to determine the presence and/or quantity of an inflammatory molecule in a subject. Exemplary MS analyses that can be used in accordance with the present invention include, but are not limited to: liquid chromatography-mass spectrometry (LC-MS); matrix-assisted laser desorption/ionization time-of-flight MS analysis (MALDI-TOF-MS), such as for example direct-spot MALDI-TOF or liquid chromatography MALDI-TOF mass spectrometry analysis; electrospray ionization MS (ESI-MS), such as for example liquid chromatography (LC) ESI-MS; and surface enhanced laser desorption/ionization time-of-flight mass spectrometry analysis (SELDI-TOF-MS). Each of these types of MS analysis can be accomplished using commercially-available spectrometers, such as, for example, triple quadropole mass spectrometers. Methods for utilizing MS analysis to detect the presence and quantity of peptides, such as inflammatory molecules, in biological samples are known in the art. See, e.g., U.S. Pat. Nos. 6,925,389; 6,989,100; and 6,890,763 for further guidance, each of which are incorporated herein by this reference.

In yet another embodiment of the therapeutic methods described herein, administering an effective amount of a composition of the present invention to the subject reduces an amount of oxidation of a low-density lipoprotein (LDL) in the subject. Current research indicates that an abundance of reactive oxygen species in the vasculature of a subject, such as what is observed in many subjects with an RAAS-related disorder results in an increased oxidation of proteins such as oxidized LDL (ox-LDL), which then initiates an inflammatory process and causes intimal damage to the arterial wall (32). While the mechanisms of this damage are not yet established and may involve the inactivation of nitric oxide (NO) by oxygen-derived free radicals such as superoxide (33), the inflammatory response seen in these subjects has been observed to affect the gene expression of various inflammatory molecules, such as VCAM and tumor necrosis factor-alpha (TNF-α; 34-36), which in turn can regulate the inflammatory process and promote foam cell formation. The reduction in NO levels along with an increase in ox-LDL may function as immunomodulators of the atherosclerotic process (37). Disclosed herein are data, however, that show that by administering a composition of the present invention to a subject affected with an RAAS-related disorder, the amount of LDL oxidation in the subjects can be significantly reduced.

Various methods of measuring an amount of LDL oxidation are known to those of ordinary skill in the art and can be used in accordance with the present invention. For example, in certain embodiments, an amount of LDL oxidation can be measured by obtaining plasma sample from subjects, isolating the LDLs by ultracentrifugation, and then oxidizing the LDL to ox-LDL using a standard assay involving CuSO₄ (52). The lag time of oxidation, which indicates the susceptibility of LDL to oxidize, can then be measured using a spectrophotometer to allow the amounts of LDL oxidation occurring in a subject to be ascertained.

Further provided, in a specific embodiment of the present invention, is a method for treating a metabolic syndrome-related disorder in a subject. In one preferred embodiment, a method for treating a metabolic syndrome-related disorder is provided that comprises administering to a subject an effective amount of a composition of the present invention, which includes an angiotensin II inhibitor and a lipoic acid compound of Formulas (I) and (II), to thereby treat the metabolic syndrome related-disorder.

As described herein above, metabolic syndrome can be considered an RAAS-related disorder as a majority of the characteristics of metabolic syndrome can be mediated by the RAAS, including abdominal obesity, dyslipidemia, elevated blood pressure, insulin resistance (i.e., impaired glucose intolerance), and pro-thrombotic and pro-inflammatory states. It has been discovered, however, that by administering a composition of the present invention to a subject in need of treatment for metabolic syndrome, the compositions of the present invention are capable of effectively treating many of the components that give rise to a diagnosis of metabolic syndrome. As such, in some embodiments of the methods of treating metabolic syndrome-related disorders disclosed herein, the metabolic syndrome-related disorder is selected from the group consisting of obesity, hypertension, impaired glucose tolerance, and dyslipidemia. When the method of treating metabolic syndrome is practiced in a subject in need, the subject is preferably administered an amount of the composition that is effective to treat the particular metabolic syndrome-related disorder being targeted. As indicated above, the effective amount for any particular subject will vary based on the subject's circumstances, and such amounts would be readily determined by one of ordinary skill in the art. For additional guidance regarding metabolic syndrome, see, e.g., Executive Summary of the Third Report of the National Cholesterol Education Program (NCEP) Expert Panel on Detection, Evaluation, and Treatment of High Blood Cholesterol in Adults (Adult Treatment Panel III). JAMA 2001; 285:2486-2497; the criteria for metabolic syndrome established by the World Health Organization; and Grundy S M. JAMA. 2003; 290(22):3000-3002, each of which is incorporated herein by this reference.

In another specific embodiment of the present invention, a method of improving vasodilation is provided whereby a subject in need of treatment is administered an amount of a composition in accordance with the invention that is effective to improve vasodilation in the subject. In some embodiments, the vasodilation is flow-mediated vasodilation, wherein the amount of vasodilation observed is related to the amount of blood flowing through a particular blood vessel. Various methods of measuring the extent of vasodilation in a subject can be used in accordance with the present invention, including the ultrasound techniques described herein above. Once again, the effective amount of a therapeutic composition administered to a subject in accordance with the present invention to improve vasodilation will vary depending on the subject's circumstances and the desired result to be achieved, but can readily be determined using routine experimentation.

In yet another specific embodiment of the present invention, a method of reducing proteinuria in a subject is provided that comprises administering to a subject in need of treatment an amount of a composition of the present invention that is effective to achieved the desired reduction in proteinuria in the subject. As will be recognized by those skilled in the art, the qualitative and quantitative measurement of proteinuria (i.e., an excess of serum proteins, such as albumin, in the urine) is an effective tool for assessing renal function in RAAS-related disorders, such as diabetes mellitus and hypertension. It has been discovered that by administering a composition of the present invention to subject, the compositions are capable of effectively reducing not only an amount of urinary albumin in the subject, but also the ratio of urinary albumin to serum creatinine in the subject. As such, in some embodiments of the presently-disclosed methods of reducing proteinuria, the reduction in proteinuria is obtained by reducing an amount of urinary albumin, reducing a ratio of urinary albumin to serum creatinine, or both. In some embodiments, proteinuria is reduced in the subject by about 10%, about 20%, about 25%, about 30%, about 35%, about 40%, about 45%, about 50%, about 55%, about 60%, about 65%, about 70%, about 75%, about 80%, about 90%, or about 99%. In some embodiments, the reduction in proteinuria is about 25% to about 75%. Again, the effective amount of a composition required to reduce proteinuria to a desired level in a particular subject will vary based on the subject's circumstances, and can be readily determined by one of ordinary skill in the art.

In still another preferred embodiment of the present invention, a method of reducing insulin resistance in a subject is provided that comprises administering an effective amount of a composition of the present invention to the subject to thereby reduce insulin resistance. Without wishing to be bound by any particular theory, it is thought that insulin resistance can play an important role in many RAAS-related disorders, and, more particularly can play a role in the hyperglycemic states that are observed in subjects with type II diabetes, which can eventually induces the development of diabetic microangiopathy (20). Furthermore, insulin resistance is proposed to play important roles in the pathogenesis of cardiovascular diseases (23,24), and is the most common cause of death in diabetic patients. It has been discovered though, that by administering a composition of the present invention to a subject with an RAAS-related disorder, the extent of overall insulin resistance in those subjects can be significantly improved, while also improving insulin receptor sensitivity. As such, in some embodiments, reducing insulin resistance comprises increasing insulin receptor sensitivity. In some embodiments, insulin resistance is reduced in the subject by about 10%, about 20%, about 25%, about 30%, about 35%, about 40%, about 45%, about 50%, about 55%, about 60%, about 65%, about 70%, about 75%, about 80%, about 90%, or about 99%. In some embodiments, the reduction in insulin resistance is about 25% to about 75%. Once again, the effective amount of a composition required to reduce insulin resistance to a desired level in a particular subject will vary based on the subject's circumstances, and can be readily determined by one of ordinary skill in the art.

The extent of insulin resistance in a given subject can be measured by a variety of methods known to those skilled in the art using surrogate indices of insulin resistance in comparison with the index assessed by euglycemic-hyperinsulinemic clamp (clamp-IR); for example, fasting plasma insulin (25), homeostasis model assessment (HOMA) of insulin resistance (HOMA-IR) (26), and the fasting glucose-to-insulin ratio (27). Indeed, it has been established that HOMA-IR is a useful surrogate index of insulin resistance in both diabetic and non-diabetic subjects and that its logarithmic transformation makes the index more accurate (28-30). Accordingly, each of the foregoing methods, including HOMA-IR, can be used in accordance with the present invention to provide an accurate assessment of insulin resistance in a given subject.

With regard to the various therapeutic methods described herein, although certain embodiments of the methods disclosed herein only call for a qualitative assessment (e.g., the presence or absence of the expression of an inflammatory gene in a subject), other embodiments of the methods call for a quantitative assessment (e.g., an amount of reduction of proteinuria in a subject or an amount of reduction of insulin resistance). Such quantitative assessments can be made, for example, using one of the above mentioned methods, as will be understood by those skilled in the art.

The skilled artisan will also understand that measuring a reduction in the amount of a certain feature (e.g., proteinuria) in a subject is a statistical analysis. For example, a reduction in an amount of proteinuria in a subject can be compared to control level of proteinuria, and an amount of proteinuria of less than or equal to the control level can be indicative of a reduction in the amount of proteinuria, as evidenced by a level of statistical significance. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Preferred confidence intervals of the present subject matter are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

The compositions of the present invention are designed to include the beneficial properties of RAAS inhibitors with those of the lipoic acid compounds described herein with reference to Formulas (I) and (II). As such, it is believed that the presently-disclosed compositions will be useful as potent antioxidants, anti-inflammatory compounds, and as mitochondrial protective agents. Consequently, it is thus further contemplated that the presently-disclosed compounds can be useful for the treatment of a number of RAAS-related disorders where a reduction in angiotensin II activity or the beneficial properties of lipoic acid are indicated.

For example, it is contemplated that the present compositions will be particularly useful in the treatment of diabetes. In this regard, it is contemplated that the compositions of the present invention will be useful for reducing oxidative stress, improving insulin signaling, treating diabetic complications that occur from overproduction of reactive oxygen and nitrogen species, and preventing the age-dependent development of hyperglycemia, hyperinsulinemia, dyslipidemia, and plasma markers of oxidative stress. Furthermore, it is also contemplated that the present compositions will be useful for preventing the mitochondrial decay that has been postulated to account for a considerable portion of the metabolic dysfunction that occurs in diabetes.

As another example, it is also contemplated that the present compositions will be useful for treating target organ damage that accompanies various RAAS-related disorders, such as hypertension, myocardial infarction, stroke, atherosclerosis, and diabetes. In this regard, it is contemplated that the present compounds will be capable of improving endothelial dysfunction by, for example, improving endothelium-dependent vasorelaxation, reducing adhesion molecules and chemokines, lowering serum triglycerides, and lowering inflammatory gene expression. In addition, it is contemplated that the present compositions will be capable of improving renal function and/or slowing the deterioration of kidney function in diabetes and hypertension by, for example, reducing or preventing the progression of microalbuminuria to subsequent overt proteinuria and renal failure.

In yet a further application of the present invention, it is contemplated that the lipoic acid compounds described herein will be present in embodiments of the compositions wherein the lipoic acid compounds further incorporate NO groups. In this regard, it is contemplated that those compositions will be useful in treating angina by making NO molecules available to the endothelium for vasodilation, thereby reversing or inhibiting coronary vasospasms that may occur in a subject.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The embodiments of the presently-disclosed subject matter as set forth herein are subject to modifications, and other modified embodiments within the scope of the invention will be evident to those of ordinary skill in the art after a study of the information provided in this document. The information provided in this document, and particularly the specific details of the described exemplary embodiments, is provided primarily for clearness of understanding and no unnecessary limitations are to be understood therefrom.

Further, while the terms used in the application are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the present invention belongs. Although any methods, devices, and materials similar or equivalent to those described herein can be used in the practice or testing of the presently-disclosed subject matter, representative methods and materials have been described herein above.

Additionally, following long-standing patent law convention, the terms “a”, “an”, and “the” refer to “one or more” when used in this application, including the claims. Thus, for example, reference to “an angiotensin-converting enzyme” includes a plurality of such enzymes, and so forth. Also, unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the present invention.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods.

### EXAMPLES

The following examples are provided which exemplify aspects of the preferred embodiments of the present invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Experimental Design and Effects of Combination Therapy on Hypertension

To evaluate the potential for co-administering a RAAS inhibitor and a lipoic acid compound for the treatment of renin-angiotensin aldosterone system-related disorders, men and women aged 18 years or older with Type II diabetes mellitus and a history of hypertension (defined as on medical therapy or systolic blood pressure greater than 140 mm Hg at the time of screening for purposes of the study) were enrolled in a randomized, crossover study employing quinapril, an angiotensin-converting enzyme (ACE) inhibitor, and alpha lipoic acid. Subjects were excluded if they had any of the following: a clinical history of coronary artery disease or congestive heart failure; use of an antihypertensive agent during the 12 months prior to enrollment; previous hypoglycemic therapy, current antihypertensive therapy, hemoglobin A₁C greater than 7.0%; serum creatinine greater than 2.0 mg/dL; hepatic impairment; or malignancy. Subjects with hypertension requiring therapy were excluded from the study as one of the goals of the study was to measure the anti-inflammatory effects of an ACE inhibitor independently of its blood pressure lowering effects. Subjects on lipid lowering therapy (e.g., statin therapy) at the time of enrollment continued their therapy without change throughout the study. Written informed consent was obtained from all subjects.

All subjects were evaluated at the time of enrollment. The subjects received nutritional counseling and were advised to record their weight and calorie counts weekly. A nutritionist was available for counseling throughout the study. Blood samples were drawn before and at the end of therapy at a similar time of day while subjects were fasting.

Subsequent to enrollment, the subjects were randomized in a double-blinded, crossover fashion to either a quinapril (40 mg/day) group or a group that was to be administered both quinapril (40 mg/day) and alpha lipoic acid (600 mg/day, Jarrow Formula, Los Angeles, Calif.) for 8 weeks. After the initial 8 weeks of treatment, there was a 4 week washout period. The subjects then received in a crossover fashion the alternate pharmacological regimen. Allocation concealment was maintained until the end of the study. Pill counts were obtained at the end of the treatment period to determine compliance (13). Subjects were advised to self-administer half of the full dose during the initial 2 days of therapy, after which they were to take the full study dose at the same time each morning. The total study period was 22 weeks.

After the initial 2 weeks, blood pressure was re-checked, and blood was drawn to measure serum creatinine and potassium. Blood pressure was checked with at least three separate measurements that were taken approximately five minutes apart using an Omron sphygmomanometer.

In total, 40 subjects (18 men and 22 women) were enrolled in the study and followed for a total period of 22 weeks, with a total of 28 subjects completing the study. Follow-up was 100% complete. Baseline characteristics are denoted in Table 2, below. Of the total study population, 12 subjects (30%) were on antihypertensive therapy.

TABLE 2
Subject demographics and baseline characteristics.

| | |
|---|---|
| Women | 22 (55.0) |
| (n (%)) | |
| Age | 46.3 ± 7.9 |
| (years) | |
| Systolic Blood Pressure | 146.6 ± 11.2 |
| (mmHg) | |
| Diastolic Blood Pressure | 90.8 ± 9.9 |
| (mmHg) | |
| Body Mass Index | 29.4 ± 4.5 |
| (kg/m²) | |
| Total cholesterol | 191.2 ± 29.7 |
| (mg/dL) | |
| LDL cholesterol | 110.1 ± 20.9 |
| (mg/dL) | |
| HDL cholesterol | 49.0 ± 10.0 |
| (mg/dL) | |
| Triglycerides | 111.3 ± 24.1 |
| (mg/dL) | |
| Glucose | 121.4 ± 14.5 |
| (mg/dL) | |
| Creatinine | 1.1 ± 0.1 |
| (mg/dL) | |
| Bilirubin | 0.6 ± 0.2 |
| (mg/dL) | |

Data are means ± SD or n (%).

During the study, it was observed that there was a similar incidence of cough in the group receiving quinapril only and in the group receiving quinapril and alpha lipoic acid (Quinapril group: 14%; Quinapril plus alpha lipoic acid group: 13%). No angioedema was noted throughout the study. There was a rise in serum potassium or creatinine of greater than 20 percent in 1 out of 40 subjects in the quinapril group. Also, there was a significant reduction in systolic and diastolic blood pressure in the quinapril and the quinapril plus alpha-lipoic acid group after the follow-up period (Table 3). No subject in either group experienced hypotension (i.e., systolic BP less than 100 mm Hg) during the study. Further, there was no significant change in glycosylated hemoglobin (Hgb) from the pretreatment time period and between the two treatment arms of the study.

TABLE 3
Changes in blood pressure and glycosylated hemoglobin.

| | Systolic BP | Diastolic BP | Glycosylated Hgb |
|---|---|---|---|
| | (mmHg) | (mmHg) | (%) |
| Pretreatment | 148.5 ± 14.6 | 86.9 ± 10.3 | 7.8 ± 1.9 |
| Quinapril | 133.4 ± 11.7* | 77.9 ± 10.0* | 7.6 ± 1.5 |
| Quinapril + | 132.9 ± 12.4* | 78.3 ± 9.6* | 7.7 ± 1.1 |
| Alpha-lipoic acid | | | |

*Value differs (p < 0.05) from pretreatment.

### Example 2

### Effects of Combination Therapy on Proteinuria

To determine the effect of co-administering quinapril and alpha lipoic acid on proteinuria in subjects with Type II diabetes and hypertension, each of the subjects described in Example 1 provided a 24 hour collection of urine at the beginning and the end of the study period for each treatment arm. Upon collection of each urine sample, the urine was quickly analyzed, and protein analysis was performed via a standard chemical analysis (Quest Laboratory, Scranton, Pa.).

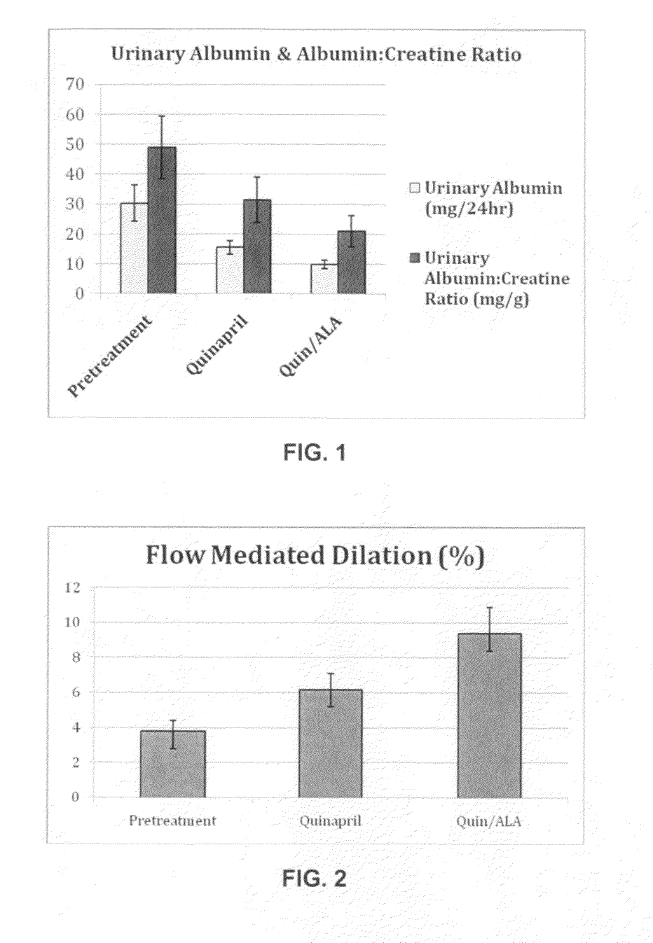

Upon analysis of the urine samples, it was observed that in subjects who were administered quinapril and in subjects who were administered the combination of quinapril and alpha lipoic acid, both urinary albumin and the ratio of urinary albumin to creatinine was reduced significantly as a result of the treatments (FIG. 1). Moreover, it was observed that the combination of quinapril and alpha lipoic acid reduced the ratio of urinary albumin to creatinine by a further 41% over quinapril alone, indicating that the combination has a significant positive effect in slowing the deterioration of kidney function in diabetes and hypertension.

### Example 3

### Effects of Combination Therapy on Endothelial Function

To determine the effect of co-administering quinapril and alpha lipoic acid on endothelial function in subjects with Type II diabetes and hypertension, an evaluation of endothelial function was performed for each of the subjects described in Example 1 by noninvasive, brachial artery reactivity testing (BART), which uses ultrasound to evaluate endothelium-dependent flow-mediated vasodilation (FMD) in the brachial artery (44). Briefly, the subjects were positioned in the supine position with the arm in a comfortable position for imaging the brachial artery. A blood pressure cuff was then placed on the forearm, after which a baseline rest image was acquired. The brachial artery was imaged above the antecubital fossa in the longitudinal plane, and a segment with clear anterior and posterior intimal interfaces between the lumen and vessel wall was selected for continuous 2D gray-scale imaging. Blood flow velocity was estimated by time-averaging the pulsed Doppler velocity signal obtained from a mid-artery sample volume. The cuff was then inflated to greater than or equal to 50 mm Hg above systolic blood pressure to occlude arterial flow for 5 minutes. After cuff deflation, the longitudinal image of the artery was recorded continuously from 30 seconds before to 2 minutes after cuff deflation. A mid-artery pulsed Doppler signal was obtained on immediate cuff release and no later than 15 seconds after cuff deflation to assess hyperemic velocity. After 15 minutes, nitroglycerin 0.4 mg was given sublingually, and repeat images were obtained to determine endothelium-independent vasodilation.

The diameter of the brachial artery was measured from longitudinal images in which the lumen-intima interface was visualized on both the near (anterior) and far (posterior) walls. Once the image for analysis was chosen, the boundaries for diameter measurements were identified manually with electronic calipers (Medical Imaging Application Vascular Tools, Coralville, Iowa), and the average diameter was determined from at least 3 different diameter measurements determined along a segment of the vessel. Brachial artery diameter was measured at the same time in the cardiac cycle by use of electrocardiogram (ECG) gating during image acquisition. FMD was typically measured as the change in post-stimulus diameter as a percentage of the baseline diameter. In accordance with established guidelines, baseline diameter, absolute change, and percent change in diameter were measured and reported (44).

Upon analysis of the results, it was observed that when the subjects were treated with quinapril only, there was a significant increase of 59 percent in flow mediated dilation of the brachial artery at 24 weeks as compared to baseline (pretreatment: 3.86±0.55 percent; Quinapril: 6.02±0.80 percent, p<0.005 Quinapril group versus pretreatment group), suggesting a trend in improvement in endothelial function (FIG. 2). Moreover, when the subjects were co-administered quinapril and alpha lipoic acid, there was a further substantial increase in endothelial function by 43 percent at the end of the 8 week treatment period (p<0.001 vs. baseline and Quinapril alone). This later finding indicated that the combination of quinapril plus alpha lipoic acid has an additive effect on the improvement of insulin receptor sensitivity in diabetic subjects with Stage I hypertension (FIG. 2).

### Example 4

### Effects of Combination Therapy on Serum Levels of Anti-Inflammatory Molecules

To determine the effect of co-administering quinapril and alpha lipoic acid on serum levels of inflammatory molecules in subjects with Type II diabetes and hypertension, plasma samples were obtained from each of the subjects described in Example 1, and were centrifuged and stored at −80° C. An aliquot of each sample was then drawn, and an enzyme immunoassay (EIA; Cayman Chemical, Ann Arbor, Mich.) for serum adiponectin and leptin was performed on each sample in triplicate according to well-established protocols (45). A total of 50 μl of serum were used for the analysis. The levels of total serum adiponectin and leptin were determined on a plate reader at an optical density of 420 nm. No interference by quinapril or its metabolites was found in any of the assays.

Upon analysis of the results, it was determined that the co-administration of quinapril and alpha lipoic acid to diabetic subjects with hypertension reduced serum levels of leptin by nearly 70% from pretreatment and that serum levels of leptin were also significantly reduced in the group of subjects taking quinapril only (Table 4). Treatment with either quinapril or with quinapril and alpha lipoic acid also significantly increased serum levels of adiponectin over pretreatment. These findings indicate that the addition of lipoic acid has a further additive and beneficial effect on markers of inflammation.

TABLE 4
Effects of quinapril and a combination quinapril and alpha lipoic
acid on serum leptin and adiponectin levels.

| | Leptin (ng/ml) | Adiponectin (ng/ml) |
|---|---|---|
| Pretreatment | 100% of | 100% of |
| | pretreatment | pretreatment |
| Quinapril | 51% of | 122% of |
| | pretreatment* | pretreatment* |
| Quinapril + | 30% of | 124% of |
| Alpha-lipoic acid | pretreatment*# | pretreatment* |

*value differs (p < 0.05) from pretreatment.
#value differs (p < 0.05) from quinapril.

### Example 5

### Effects of Combination Therapy on Insulin Resistance

Without wishing to be bound by any particular theory, it was thought that insulin resistance can play an important role in hyperglycemia in type 2 diabetic subjects, and can eventually induce the development of diabetic microangiopathy (20). Indeed, to achieve glycemic control and prevent these complications, several oral hypoglycemic agents that improve insulin resistance, such as thiazolidinediones and biguanides, have been developed and are currently available clinically (21,22). Furthermore, insulin resistance is proposed to play important roles in the pathogenesis of cardiovascular diseases (23,24), the most common cause of death in diabetic subjects. Therefore, a clinical and epidemiological evaluation was undertaken with the subjects described in Example 1 in order to evaluate insulin resistance simply and accurately in the individual diabetic subjects with hypertension. Many investigators have studied simple surrogate indices of insulin resistance in comparison with the index assessed by euglycemic-hyperinsulinemic clamp (clamp-IR); for example, fasting plasma insulin (25), homeostasis model assessment (HOMA) of insulin resistance (HOMA-IR) (26), and the fasting glucose-to-insulin ratio (27). Further, it has been established that HOMA-IR is a useful surrogate index of insulin resistance in diabetic and non-diabetic subjects and that its logarithmic transformation makes it more accurate (28-30). As such, to determine the effect of co-administering quinapril and alpha-lipoic acid on insulin resistance in subjects with Type II diabetes and hypertension, a HOMA-IR index of insulin resistance was established according to well-established protocols.

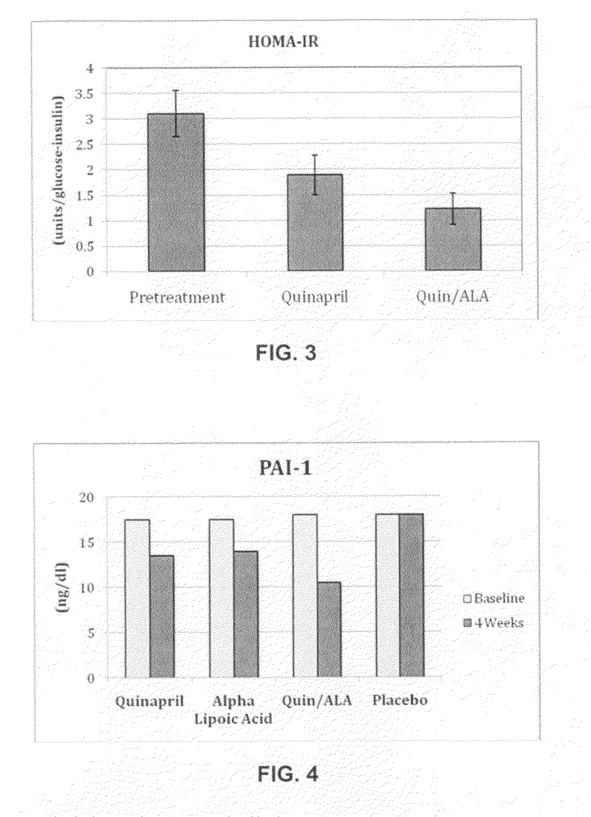

Upon analysis of the results from the HOMA-IR evaluation, it was observed that in the quinapril only treatment arm, there was a significant reduction of 40% from pretreatment baseline in serum HOMA-IR (pretreatment: 3.01±0.33 U/ml; Quinapril: 1.83±0.25 U/ml, p<0.005 Quinapril versus pretreatment). Furthermore, when subjects were in the quinapril and alpha lipoic acid treatment arm, the findings were statistically significant as compared to the quinapril only group at the end of the treatment period (Quinapril and alpha-lipoic acid group: 1.26±0.14 U/ml, p<0.005 Quinapril and alpha lipoic acid group versus pretreatment and quinapril groups, FIG. 3). These findings thus revealed that co-administration of quinapril and alpha lipoic acid allowed a reduction in the HOMA-IR index, determined by calculation (31), by nearly 70% (FIG. 3). Further, as evidenced by the marked reduction in the HOMA-IR index from pretreatment and the significant difference from the group receiving quinapril only, the results obtained indicate that the combination therapy not only improves insulin receptor sensitivity, but also reduces overall insulin resistance.

### Example 6

### Effects of Combination Therapy on Low-Density Lipoprotein Oxidation

Current research indicates that an abundance of reactive oxygen species in the vasculature results in increased oxidation of proteins including oxidized low-density lipoproteins (ox-LDL), which initiate an inflammatory process and cause intimal damage to arterial walls (32). Although, the mechanisms of this damage have yet to be clearly established and may involve the inactivation of nitric oxide (NO) by oxygen-derived free radicals such as superoxide (33), it is clear that this inflammatory response affects the gene expression of regulatory molecules, such as vascular cell adhesion molecule and tumor necrosis factor-alpha (34-36), which in turn promote foam cell formation. In this regard, a reduction in NO levels along with an increase in ox-LDL may function as immunomodulators of the atherosclerotic process (37) and, indeed, recent studies imply that ox-LDL stimulates an immunological response through the formation of autoantibodies, resulting in further damage to the endothelium and acceleration of the atherosclerotic process (38,39). This antibody response may represent a marker for the extent of atherosclerosis seen in individuals. As such, to determine the effect of co-administering quinapril and alpha-lipoic acid on levels of ox-LDLs and gain insight into potential inflammatory responses occurring in subjects with Type II diabetes and hypertension, plasma samples were obtained and isolated from certain of the subjects described in Example 1 (i.e., subjects on quinapril who crossed over to quinapril and alpha lipoic acid), and LDL was isolated via ultracentrifugation at 39,000 rpm at 4° C. The LDL was then oxidized to ox-LDL by an in vitro assay utilizing CuSO₄ (52). The lag time indicating the susceptibility of the LDL to oxidize was measured using a spectrophotometer at 280 nm (42). Values were performed in triplicate.

Using a time course analysis, upon analysis of the results from these experiments, it was observed that the co-administration of quinapril and alpha lipoic acid, as well as the administration of quinapril only, increased the lag time of LDL oxidation in these subjects (Table 5) with a 23 percent increase from pretreatment in the quinapril arm (p<0.005 from pretreatment) and a 44 percent increase from pretreatment in the quinapril and alpha lipoic acid arm (p<0.005 from pretreatment, p=0.041 from quinapril). These findings thus indicate that co-administering quinapril and alpha lipoic acid has a significant antioxidant effect within the vasculature.

TABLE 5
Effects of quinapril and combination quinapril and alpha-lipoic
acid on LDL oxidation in patients with diabetes and hypertension.

| | Pretreatment | Post-treatment |
|---|---|---|
| | (sec) | (sec) |
| Quinapril | 58.5 ± 10.0 | 71.0 ± 13.9* |
| Quinapril + | 57.2 ± 13.5 | 82.3 ± 14.2*# |
| Alpha-lipoic acid | | |

*Value differs (p < 0.05) from pretreatment.
#Value differs (p < 0.05) from quinapril.

### Example 7

### Effect of Combination Therapy in Subjects with Metabolic Syndrome

To determine the effect of co-administering quinapril and alpha lipoic acid to subjects with metabolic syndrome, subjects with metabolic syndrome and a family history of premature coronary artery disease were identified and enrolled in a study. In the study, the subjects were randomized in a double-blinded fashion to the following treatment groups: placebo; quinapril (20 mg/day), alpha lipoic acid (300 mg/day), or quinapril (20 mg/day) and alpha lipoic acid (300 mg/day). The therapeutic agents were administered in separate pills for a 12 week period, and the patients were followed at 6 and 12 weeks of therapy. Blood was collected at these time periods, and serum levels of soluble PAI-1 and VCAM-1 were determined using an enzyme-linked immunosorbent assay (ELISA). Further, endothelial function in each of the subjects was also determined by flow mediated dilation (FMD) of the brachial artery, utilizing the high resolution ultrasound technique described herein above.

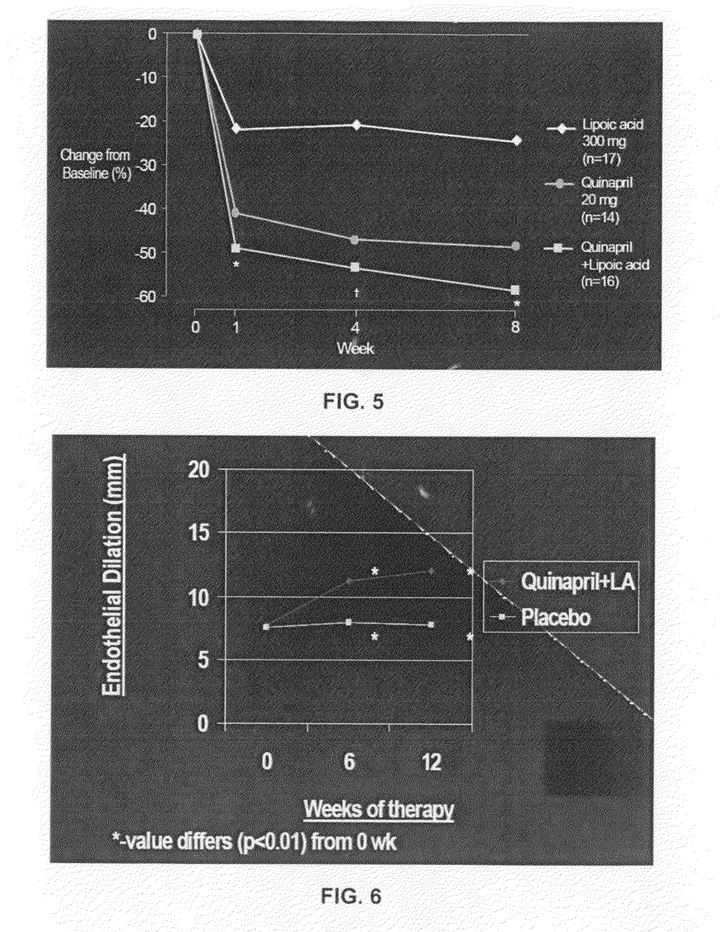

Upon analysis of the results from this study, it was observed that co-administering quinapril (20 mg/day) and alpha lipoic acid (300 mg/day) decreased serum levels of the inflammatory markers PAI-1 and VCAM-1 (FIG. 4 and FIG. 5, respectively) in the subjects. In particular, after four weeks of therapy, serum PAI-1 levels (ng/dl) were reduced by 22%, 21%, and 40% in the quinapril, lipoic acid, and quinapril/lipoic acid treatments groups, respectively (see FIG. 4; p<0.01 from baseline; p<0.01 from quinapril or lipoic acid). Furthermore, the co-administration of quinapril and alpha lipoic acid also markedly improved endothelial function in the subjects (FIG. 6; (*) p<0.01 from baseline of 0 weeks). Taken together, these results thus demonstrate a significant improvement in levels of inflammation and in endothelial function in subjects with metabolic syndrome and a family history of coronary artery disease.

### Example 8

### Effect of Combination Therapy on Stroke

To assess the effects of co-administering alpha lipoic acid and the ACE inhibitor captopril on stroke, normal Sprague-Dawley rats were first pretreated with either saline alone or specific amounts of a composition where 5 mg of alpha-lipoic acid was combined with 0.5 mg of captopril. The animals in the test group were divided into two separate subgroups, with the first subgroup receiving 1 mg/kg body weight of the composition and the second subgroup receiving 5 mg/kg body weight of the composition. An acute cerebral infarct was then induced in all groups by occlusion of the cerebral arteries in the rats. Subsequent to occlusion, the size of the cerebral infarct in each of the rats was then assessed via phosphoimaging quantification. During the experiments, blood pressure was also recorded in the tail vain of each of the rats.

Upon analysis of the results of these experiments, it was observed that co-administration of alpha lipoic acid and captopril effectively reduced the cerebral tissue damage. In particular, co-administration of alpha-lipoic acid and captopril significantly reduced the volume of the infarct at doses of 1 mg/kg and 5 mg/kg without significantly affecting the blood pressure in the rats (Table 6). As such, the foregoing results thus indicate that co-administration of a composition comprising an effective amount of alpha-lipoic acid and captopril can effectively be used in a method of treating stroke.

TABLE 6
Effect of a combination of alpha-lipoic acid and captopril on
cerebral infarct size and systolic blood pressure.

| | Infarct volume | Systolic BP |
|---|---|---|
| | (mm³) | (mmHg) |
| Vehicle | 28.4 ± 5.3 | 161 ± 39 |
| (n = 8) | | |
| Captopril and | 12.9 ± 2.7 | 158 ± 29 |
| alpha lipoic acid | | |
| (1 mg/kg, n = 4) | | |
| Captopril and | 10.8 ± 2.3 | 151 ± 32 |
| alpha lipoic acid | | |
| (5 mg/kg, n = 4) | | |

Throughout this document, various references are mentioned. All such references are incorporated herein by reference, including the references set forth in the following list:

### REFERENCES

1. De Carvalho Frimm C et al. Angiotensin II receptor blockade and myocardial fibrosis of infarcted rat heart. J Lab Clin Med 1997 April; 129 (4): 439-46.
2. Mahmud A, Feely J. Effect of angiotensin II receptor blockade on arterial stiffness: beyond blood pressure reduction. Am J Hypertension 2002; 15: 1092-1095.
3. Verma S, Buchanan M, Anderson T. Endothelial function as a biomarker of vascular disease. Circulation 2003; 108:2054-2059.
4. Corretti M C, Anderson T J, Benjamin E J, Celermajer D, Charbonneau F, Creager M A, Deanfield J, Drexler H, Gerhard-Herman M, Herrington D, Valiance P, Vita J, Vogel R. Guidelines for the Ultrasound Assessment of Endothelial-Dependent Flow-Mediated Vasodilation of the Brachial Artery. J Am Coll Card 2002; 39(2): 257-265.
5. Anderson T J, Uehata A, Gerhard M D, Meredith I T, Knab S, Delagrange D, Lieberman E H, Ganz P, Creager M A, Yeung A C. Close relation of endothelial function in the human coronary and peripheral circulations. J Am Coll Cardiol 1995; 26:1235-1241.
6. Rosamond W, Flegal K, Friday G, Furie K, Go A, Greenlund K, Haase N, Ho M, Howard V, Kissela B, Kittner S, Lloyd-Jones D, McDermott M, Meigs J, Moy C, Nichol G, O'Donnell C J, Roger V, Rumsfeld J, Sorlie P, Steinberger J, Thom T, Wasserthiel-Smoller, S, Hong Y for the American Heart Association Statistics Committee and Stroke Statistics Subcommittee Heart disease and stroke statistics—2007 update: a report from the American Heart Association Statistics Committee and Stroke Statistics Subcommittee. Circulation. 2007; 115(5):e69-171.
7. Cooper R, Rotimi C. Hypertension in blacks. Am J Hypertens. 1997; 10(7 Pt 1) 804-812.
8. Ong K L, Cheung B M, Man Y B, Lau C P, Lam K S. Prevalence, awareness, treatment, and control of hypertension among United States adults 1999-2004. Hypertension. 2007; 49(1):69-75.
9. Lawes C M, Vander Hoorn S, Law M R, Elliott P, MacMahon S, Rodgers A. Blood pressure and the global burden of disease 2000. Part 1: estimates of blood pressure levels. J Hypertens. 2006; 24(3):413-422.
10. Ferdinand K C. Cardiovascular disease and African Americans: why determination of race is inadequate for research and practice. J Natl Med Assoc. 2007; 99(6):686-689.
11. Hong W, Lai H, Yang C, Ren S, Dai S, Lai S. Age, gender and metabolic syndrome-related coronary heart disease in U.S. adults. Int J Cardiol. 10 2005; 104(3):288-291.
12. Lakka H M, Laaksonen D E, Lakka T A, Niskanen L K, Kumposalo E, Salonen J T. The metabolic syndrome and total and cardiovascular disease mortality in middle-aged men. JAMA 2002; 288(21):2709-2716.
13. Grundy S M. Inflammation, hypertension, and the metabolic syndrome. JAMA. 2003; 290(22):3000-3002.
14. Chandran M, Phillips S A, Ciaraldi T, Henry R R. Adiponectin: more than just another fat cell hormone? Diabetes Care. August 2003; 26(8):2442-2450.
15. Cote M, Mauriege P, Bergeron J. Adiponectinemia in visceral obesity: impact on glucose tolerance and plasma lipoprotein and lipid levels in men. J Clin Endocrinol Metab. March 2005; 90(3):1434-1439.
16. Schulze M B, Shai I, Rimm E B, Li T, Rifai N, Hu F B. Adiponectin and future coronary heart disease events among men with type 2 diabetes. Diabetes. February 2005; 54(2):534-539.
17. Ouchi N, Kihara S, Arita Y, Okamoto Y, Maeda K, Kuriyama H, et al. Adiponectin, an adipocyte-derived plasma protein, inhibits endothelial NF-kappaB signaling through a cAMP-dependent pathway. Circulation. 2000; 102(11):1296-1301.
18. Zhang Y, Proenca R, Maffei M, Barone M, Leopold L, Friedman J M. Positional cloning of the mouse obese gene and its human homologue. Nature. 1994; 372(6505):425-432.
19. Lord G M, Matarese G, Howard J K, Baker R J, Bloom S R, Lechler R I. Leptin modulates the T-cell immune response and reverses starvation-induced immunosuppression. Nature. Aug. 27, 1998; 394(6696):897-901.
20. Reaven G M: Banting Lecture 1988: role of insulin resistance in human disease. Diabetes 37: 1595-1607, 1988
21. Saltiel A R, Olefsky J M: Thiazolidinediones in the treatment of insulin resistance and type II diabetes. Diabetes 45: 1661-1669, 1996
22. Garber A J, Duncan T G, Goodman A M, Mills D J, Rohlf J L: Efficacy of metformin in type II diabetes: results of a double-blind, placebo-controlled, dose-response trial. Am J Med 103: 491-497, 1997
23. DeFronzo R A: Lilly Lecture 1987: the triumvirate: beta-cell, muscle, liver: a collusion responsible for NIDDM. Diabetes 37: 667-687, 1988
24. DeFronzo R A, Tobin J D, Andres R: Glucose clamp technique: a method for quantifying insulin secretion and resistance. Am J Physiol 237: E214-E223, 1979
25. Laakso M: How good a marker is insulin level for insulin resistance? Am J Epidemiol 137: 959-965, 1993
26. Matthews D R, Hosker J P, Rudenski A S, Naylor B A, Treacher D F, Turner R C: Homeostasis model assessment: insulin resistance and beta-cell function from fasting plasma glucose and insulin concentrations in man. Diabetologia 28: 412-419, 1985
27. Legro R S, Finegood D, Dunaif A: A fasting glucose to insulin ratio is a useful measure of insulin sensitivity in women with polycystic ovary syndrome. J Clin Endocrinol Metab 83: 2694-2698, 1998
28. Emoto M, Nishizawa Y, Maekawa K, Hiura Y, Kanda H, Kawagishi T, Shoji T, Okuno Y, Morii H: Homeostasis model assessment as a clinical index of insulin resistance in type 2 diabetic subjects treated with sulfonylureas. Diabetes Care 22: 818-822, 1999
29. Bonora E, Targher G, Alberiche M, Bonadonna R C, Saggiani F, Zenere M B, Monauni T, Muggeo M: Homeostasis model assessment closely mirrors the glucose clamp technique in the assessment of insulin sensitivity: studies in subjects with various degrees of glucose tolerance and insulin sensitivity. Diabetes Care 23: 57-63, 2000
30. Hermans M P, Levy J C, Morris R J, Turner R C: Comparison of insulin sensitivity tests across a range of glucose tolerance from normal to diabetes. Diabetologia 42: 678-687, 1999
31. Katz A, Nambi S S, Mather K, Baron A D, Follmann D A, Sullivan G, Quon M J: Quantitative insulin sensitivity check index: a simple, accurate method for assessing insulin sensitivity in humans. J Clin Endocrinol Metab 85: 2402-2410, 2000
32. Witztum J L, Steinberg D. Role of oxidized low density lipoprotein in atherogenesis. J Clin Invest 1991; 88:1785-1792.
33. Rajagopalan S, Harrison D G. Reversing endothelial dysfunction with angiotensin-converting enzyme inhibitors. A new trend? Circulation 1996; 94:240-243.
34. Henninger D D, Gerritsen M E, Granger D N. Low-density lipoprotein receptor knockout mice exhibit exaggerated microvascular responses to inflammatory stimuli. Circ Res 1997; 81:274-281.
35. Stannard A K, Khan S, Graham A, Owen J S, Allen S P. Inability of plasma high-density lipoproteins to inhibit cell adhesion molecule expression in human coronary artery endothelial cells. Atherosclerosis 2001; 154:31-38.
36. Libby P, Geng Y J, Aikawa M, et al. Macrophages and atherosclerotic plaque stability. Curr Opin Lipidol 1996; 7:330-335.
37. Vergnani L, Hatrik S, Ricci F, et al. Effect of native and oxidized low-density lipoprotein on endothelial nitric oxide and superoxide production: key role of L-arginine availability. Circulation 2000; 101:1261-1266.
38. Wu J T, Wu L L. Autoantibodies against oxidized LDL. A potential marker for atherosclerosis. Clin Lab Med 1997; 17:595-604.
39. Scott B C, Arouma O I, Evans P J. Lipoic and dihydrolipoic acid as antioxidants: a critical evaluation. Free Radic Res. 1994; 20:119-133.
40. Suzuki Y J, Tsuchiya M, Packer L. Thiotic acid and dihydrolipoic acid are novel antioxidants which interact with reactive oxygen species. Free Radic Res Commun. 1991; 15:255-263.
41. Passwater R A. Lipoic Acid: The Metabolic Antioxidant. New Canaan, Conn.: Keats Publishing Inc; 1995:1-47.
42. Zieden B, Wuttge D M, Karlberg B E, Olsson A G. Effects of in vitro addition of captopril on copper-induced low density lipoprotein oxidation. Br J Clin Pharmacol 1995; 39:201-3.
43. Khan B V, Sola S, Lauten W B, Natarajan R, Hooper W C, Menon R G, Lerakis S, Helmy T. Quinapril, an ACE inhibitor, reduces markers of oxidative stress in the metabolic syndrome. Diabetes Care. 2004; 27:1712-1715.
44. Sola S, Mir M Q M, Cheema F, Merchant N, Menon R G, Parthasarathy S, Khan B V. Irbesartan and Lipoic Acid Improve Endothelial Function and Reduce Markers of Inflammation in the Metabolic Syndrome: Results of the Irbesartan and Lipoic Acid in Endothelial Dysfunction (ISLAND) Study. Circulation. 2005; 111:343-348.
45. Chobanian A V. The effects of angiotensin converting enzyme inhibitors and other antihypertensive drugs on cardiovascular risk factors and atherogenesis. Clin Cardiol. 1990; 13:VII43-VII48.
46. Scott M. Grundy, PhD, Chair, James I. Cleeman MD, Co-Chair, Stephen R. Daniels MD, PhD, Karen A. Donato MS, RD, Robert H. Eckel MD, Barry A. Franklin PhD, David J. Gordon MD, PhD, MPH, Ronald M. Krauss MD, Peter J. Savage MD, Sidney C. Smith Jr MD, John A. Spertus MD, and Fernando Costa MD. Diagnosis and Management of the Metabolic Syndrome. An American Heart Association/National Heart, Lung, and Blood Institute Scientific Statement. Circulation 2005; 112:2735.
47. Kim S, Whelan J, Claycombe K, Reath D B, Moustaid-Moussa N. Angiotensin II increases leptin secretion by 3T3-LI and human adipocytes via a prostaglandin-independent mechanism. J Nutr. 2002; 132:1135-1140.
48. Hattori Y, Akimoto K, Gross S S, Hattori S, Kasai K. Angiotensin II induced oxidative stress elicits hypoadiponectinaemia in rats. Diabetologia. 2005; 48(6): 1066-1074.
49. Furuhashi M, Ura N, Higashiura K, Murakami H, Tanaka M, Moniwa N, Yoshida D, Shimamoto K. Blockade of the Renin-Angiotensin System Increases Adiponectin Concentrations in Patients With Essential Hypertension. Hypertension. 2003; 42:76-81.
50. Candido R, Jandeleit-Dahm K A, Cao Z. Prevention of accelerated atherosclerosis by angiotensin converting enzyme inhibition I diabetic apolipoprotein E-deficient mice. Circulation. 2002; 106:246-253.
51. Zhang W, Bird K E, McMillen T S, LeBoeuf R C, Hagen T M, Frei B. Dietary α-Lipoic Acid Supplementation Inhibits Atherosclerotic Lesion Development in Apolipoprotein E-Deficient and Apolipoprotein E/Low-Density Lipoprotein Receptor-Deficient Mice. Circulation. 2008; 117: 421-428.
52. Zieden B, Wuttge D M, Karlberg B E, Olsson A G. Effects of in vitro addition of captopril on copper-induced low density lipoprotein oxidation. Br J Clin Pharmacol 1995; 39:201-203.
53. Executive Summary of the Third Report of the National Cholesterol Education Program (NCEP) Expert Panel on Detection, Evaluation, and Treatment of High Blood Cholesterol in Adults (Adult Treatment Panel III). JAMA 2001; 285:2486-2497.

It will be understood that various details of the present invention can be changed without departing from the scope of the subject matter disclosed herein. Furthermore, the foregoing description is for the purpose of illustration only, and not for the purpose of limitation.

What is claimed is:

1. A composition, comprising a renin-angiotensin aldosterone system inhibitor and a lipoic acid compound selected from the group consisting of the following Formulas (I) and (II), or pharmaceutically-acceptable salts or solvates thereof: 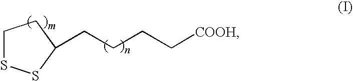
wherein:
m is an integer from 1 to 2; and n is an integer from 1 to 5;
and 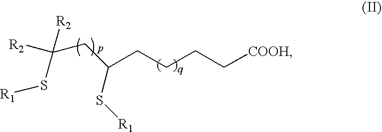
wherein:
p is an integer from 1 to 2; q is an integer from 1 to 5;
R₁ is selected from the group consisting of H, methyl, NO, and acetyl; and
R₂ is selected from the group consisting of H, methyl, and tert-butyl.

2. The composition of claim 1, wherein m is 2.

3. The composition of claim 1, wherein n is an integer from 2 to 5.

4. The composition of claim 1, wherein the renin-angiotensin aldosterone system inhibitor is selected from the group consisting of an angiotensin-converting enzyme inhibitor and an angiotensin II receptor blocker.

5. The composition of claim 4, wherein the renin-angiotensin aldosterone system inhibitor is an angiotensin-converting enzyme inhibitor selected from the group consisting of benazepril, captopril, cilazapril, enalapril, enalaprilat, fosinopril, lisinopril, moexipril, perindopril, quinapril, ramipril, trandolapril, and zofenopril.

6. The composition of claim 4, wherein the renin-angiotensin aldosterone system inhibitor is an angiotensin II receptor blocker selected from the group consisting of candesartan, eprosartan, irbesartan, telmisartan, valsartan, losartan, and olmesartan.

7. The composition of claim 1, further comprising a statin.

8. The composition of claim 7, wherein the statin is selected from the group consisting of atorvastatin, fluvastatin, lovastatin, mevastatin, pitavastatin, pravastatin, rosuvastatin, and simvastatin.

9. The composition of claim 1, further comprising an anti-inflammatory agent, an agent that inhibits absorption of fatty acids, or combinations thereof.

10. The composition of claim 1, further comprising a pharmaceutically-acceptable vehicle, carrier, or excipient.

11. The composition of claim 1, wherein the composition is in a sustained-release formulation.

12. A composition comprising a renin-angiotensin aldosterone system inhibitor, a statin, and a lipoic acid compound selected from the group consisting of the following Formulas (I) and (II), or pharmaceutically-acceptable salts or solvates thereof: 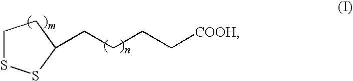
wherein:
m is an integer from 1 to 2; and n is an integer from 1 to 5;
and 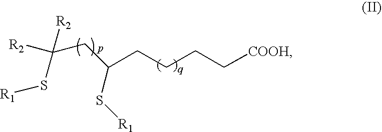
wherein:
p is an integer from 1 to 2; q is an integer from 1 to 5;
R₁ is selected from the group consisting of H, methyl, NO, and acetyl; and
R₂ is selected from the group consisting of H, methyl, and tert-butyl.

13. The composition of claim 12, wherein the renin-angiotensin aldosterone system inhibitor is selected from the group consisting of an angiotensin-converting enzyme inhibitor and an angiotensin II receptor blocker.

14. The composition of claim 13, wherein the renin-angiotensin aldosterone system inhibitor is an angiotensin-converting enzyme inhibitor selected from the group consisting of benazepril, captopril, cilazapril, enalapril, enalaprilat, fosinopril, lisinopril, moexipril, perindopril, quinapril, ramipril, trandolapril, and zofenopril.

15. The composition of claim 13, wherein the renin-angiotensin aldosterone system inhibitor is an angiotensin II receptor blocker selected from the group consisting of candesartan, eprosartan, irbesartan, telmisartan, valsartan, losartan, and olmesartan.

16. The composition of claim 12, wherein the statin is selected from the group consisting of atorvastatin, fluvastatin, lovastatin, mevastatin, pitavastatin, pravastatin, rosuvastatin, and simvastatin.

17. The composition of claim 12, further comprising an anti-inflammatory agent, an agent that inhibits absorption of fatty acids, or combinations thereof.

18. The composition of claim 12, further comprising a pharmaceutically-acceptable vehicle, carrier, or excipient.

19. The composition of claim 12, wherein the composition is in a sustained-release formulation.

20. A method of treating a renin-angiotensin aldosterone system-related disorder comprising administering to a subject in need thereof an effective amount of a composition comprising a renin-angiotensin aldosterone system inhibitor and a lipoic acid compound selected from the group consisting of the following Formulas (I) and (II), or pharmaceutically-acceptable salts or solvates thereof: 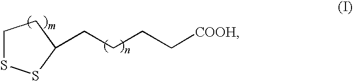
wherein:
m is an integer from 1 to 2; and n is an integer from 1 to 5; and 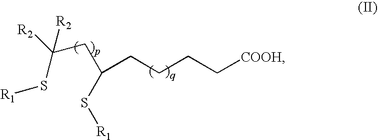
wherein:
p is an integer from 1 to 2; q is an integer from 1 to 5;
R₁ is selected from the group consisting of H, methyl, NO, and acetyl; and
R₂ is selected from the group consisting of H, methyl, and tert-butyl.

21. The method of claim 20, wherein the renin-angiotensin aldosterone system inhibitor is selected from the group consisting of an angiotensin-converting enzyme inhibitor and an angiotensin II receptor blocker.

22. The method of claim 21, wherein the renin-angiotensin aldosterone system inhibitor is an angiotensin-converting enzyme inhibitor selected from the group consisting of benazepril, captopril, cilazapril, enalapril, enalaprilat, fosinopril, lisinopril, moexipril, perindopril, quinapril, ramipril, trandolapril, and zofenopril.

23. The method of claim 21, wherein the renin-angiotensin aldosterone system inhibitor is an angiotensin II receptor blocker selected from the group consisting of candesartan, eprosartan, irbesartan, telmisartan, valsartan, losartan, and olmesartan.

24. The method of claim 20, wherein the composition further comprises a statin.

25. The method of claim 24, wherein the statin is selected from the group consisting of atorvastatin, fluvastatin, lovastatin, mevastatin, pitavastatin, pravastatin, rosuvastatin, and simvastatin.

26. The method of claim 20, wherein the composition further comprises an anti-inflammatory agent, an agent that inhibits absorption of fatty acids, or combinations thereof.

27. The method of claim 20, wherein the renin-angiotensin aldosterone system-related disorder is selected from the group consisting of hypertension, diabetes mellitus, target organ damage related to diabetes mellitus, atherosclerosis, coronary heart disease, angina, stroke, renal disorders, Reynaud's disease, metabolic syndrome, obesity, impaired glucose tolerance, and dyslipidemia.

28. The method of claim 20, wherein administering the composition to the subject increases endothelial function in a blood vessel of the subject.

29. The method of claim 20, wherein administering the composition to the subject reduces the level of an inflammatory molecule in the subject.

30. The method of claim 29, wherein the inflammatory molecule is selected from the group consisting of PAI-1, VCAM-1, leptin, and adiponectin.

31. The method of claim 20, wherein administering the composition to the subject reduces an amount of oxidation of a low-density lipoprotein in the subject.

32. The method of claim 20, wherein the subject is a mammal.

33. The method of claim 32, wherein the mammal is a human.

34. A method of improving vasodilation, comprising administering to a subject in need thereof an effective amount of the composition of claim 1.

35. The method of claim 34, wherein the vasodilation is flow-mediated vasodilation.

36. A method of reducing proteinuria, comprising administering to a subject in need thereof an effective amount of the composition of claim 1.

37. The method of claim 36, wherein proteinuria is reduced in the subject by about 25% to about 75%.

38. The method of claim 36, wherein the reduction in proteinuria is obtained by reducing an amount of urinary albumin, reducing a ratio of urinary albumin to serum creatinine, or reducing both an amount of urinary albumin and a ratio of urinary albumin to serum creatinine.

39. A method of reducing insulin resistance, comprising administering to a subject in need thereof an effective amount of the composition of claim 1.

40. The method of claim 39, wherein insulin resistance is reduced by about 25% to about 75%.

41. The method of claim 39, wherein insulin receptor sensitivity in a subject is increased.

42. A method of treating a metabolic syndrome-related disorder, comprising administering to a subject in need thereof an effective amount of the composition of claim 1.

43. The method of claim 42, wherein the metabolic syndrome-related disorder is selected from the group consisting of obesity, hypertension, impaired glucose tolerance, and dyslipidemia.
